(19) United States
(12) Patent Application Publication
(10) Pub. No.: US 2021/0345943 A1
(43) Pub. Date: Nov. 11, 2021

(54) BONE DENSITY MEASURING DEVICE AND BONE DENSITY IMAGING METHOD

(71) Applicant: Shimadzu Corporation, Kyoto (JP)

(72) Inventors: Tomohiro NAKAYA, Kyoto (JP); Daisuke NOTOHARA, Kyoto (JP); Shinya HIRASAWA, Kyoto (JP)

(21) Appl. No.: 16/870,559

(22) Filed: May 8, 2020

(51) Int. Cl. A61B 5/00; A61B 6/00; A61B 6/06; H05G 1/26; H05G 1/32; G06T 5/50

(52) U.S. Cl. CPC A61B 5/4509; A61B 6/505; A61B 6/06; G06T 5/50; A61B 6/4208; H05G 1/265; H05G 1/32; A61B 6/5205

(57) ABSTRACT

A bone density measuring apparatus and a bone density imaging method capable of improving the accuracy of a bone density analysis are provided. In a state in which no subject is present, a detector detects X-rays emitted from an X-ray tube under a high tube voltage X-ray condition/a low tube voltage X-ray condition and a first gain correction map/a second gain correction map is generated (S1, S2). A detector detects the X-rays emitted from an X-ray tube and transmitted through a subject under a high tube voltage X-ray condition/a low tube voltage X-ray condition, and a high voltage image/a low voltage image captured by the detector is generated (S3). By performing a gain correction of the high voltage image using the first gain correction map, performing a gain correction of the low voltage image using the second gain correction map (S4), and performing a subtraction of the high voltage image after the gain correction and the low voltage image after the gain correction (S5), the accuracy of the bone density analysis can be improved.

### CROSS-REFERENCE TO RELATED APPLICATIONS

The present application is related to Japanese Patent Application No. 2018-010707, filed on Jan. 25, 2018 and published on Aug. 1, 2019, the contents of which are incorporated herein by reference in its entirety.

### TECHNICAL FIELD

The present invention relates to a bone density measuring apparatus and a bone density imaging method for measuring a bone density by X-ray imaging.

### BACKGROUND OF THE INVENTION

In imaging for a bone density measurement of imaging a lumbar spine or a femur of a subject (hereinafter abbreviated as “bone density imaging”), a measurement using an X-ray beam having two different energy peaks called a DXA (Dual Energy X-Ray Absorptiometry) method (also referred to as “DEXA method”) is performed (see, for example, Patent Documents 1 and 2). Specifically, in the case of imaging a femur in a state in which a subject (patient) is laid on a top board, X-rays are perpendicularly incident on the proximal femur by pivoting the leg internally as described in Patent Document 1: WO 2017/026046. In the case of imaging a lumbar spine, X-rays are perpendicularly incident on the lumbar spine in a state in which a subject (patient) is laid on a top board and the subject's knees are bent so that the lumbar spine is in close contact with the top board.

In the bone density imaging by a DXA method, a high tube voltage X-ray condition and a low tube voltage X-ray condition and dedicated metal filters are combined in order to emit an X-ray beam having two different energy peaks. As disclosed in Patent Document 2 (Japanese Unexamined Patent Application Publication No. 2017-127342), a high tube voltage X-ray condition denotes a high voltage condition in which a high voltage is applied to an X-ray tube, and a low tube voltage X-ray condition denotes a low voltage condition in which a low voltage lower than the high voltage is applied to the X-ray tube. As disclosed in Patent Document 1 (WO 2017/026046), two types of metal filters, i.e., a metal filter for a high voltage mode and a metal filter for a low voltage mode, are provided so that the metal filters are switchably arranged on the irradiation side of the X-ray tube. When the metal filter for a high voltage mode is switched so as to be arranged on the irradiation side of the X-ray tube, the high-energy X-rays transmitted through the metal filter for a high voltage mode is emitted. When the metal filter for a low voltage mode is switched so as to be arranged on the irradiation side of the X-ray tube, the low-energy X-rays transmitted through the metal filter for a low voltage mode is emitted.

By the X-ray beam having two different energy peaks, a high voltage image captured under the high voltage condition and a low voltage image captured under the low voltage condition are generated. By subtracting these images to generate a subtraction image, only a bone, such as, e.g., a lumbar spine and a femur, is selectively reflected in the subtraction image (see, e.g., Non-Patent Document 1). The subtraction processing described in this specification includes a logarithmic conversion, weighting processing, and difference processing as described in Patent Document 2 (Japanese Unexamined Patent Application Publication No. 2017-127342) and page 22 of Non-Patent Document 1. A subtraction image is acquired by calculating the difference between the high voltage image and the low voltage image subjected to the logarithmic conversion and the weighting processing.

By the bone density imaging by a DXA method, it is possible to acquire a bone density such as a bone mineral content, which is an indicator of the amount contained in a bone of a mineral content (such as calcium and phosphorus) contained in a constant amount of a bone.

### PRIOR ART DOCUMENT

### Patent Document

Patent Document 1
International Publication No. WO 2017/026046
Patent Document 2
Japanese Unexamined Patent Application Publication No. 2017-127342

### Non-Patent Document

Non-Patent Document 1
Tatsushi Tomomitsu (Author), Teruki Sone (Author), Masao Fukunaga (Supervisor), Chihiro Yaekashi (Illustration), “Illustrative Bone Quantity Measurement by DXA—Lumbar Spine and Proximal Portion of Femur”, Life Science Publishing, Mar. 1, 2013, p. 20, 22 to 25

### SUMMARY OF THE INVENTION

### Problems to be Solved by the Invention

Since a bone density measurement is a quantitative measurement, precise bone density imaging is desired.

The present invention was made in view of the above-described circumstances, and an object of the present invention is to provide a bone density measuring apparatus and a bone density imaging method capable of improving the accuracy of a bone density analysis.

### Means for Solving the Problem

As a result of the intensive research to solve the above-mentioned problems, the inventors have acquired the following findings.

That is, attention has been paid to gain calibration and gain correction performed in common X-ray imaging. In a flat panel X-ray detector (hereinafter abbreviated as “FPD”), the gain calibration processing is performed to correct the unevenness, etc., in the sensitivity in the detection plane (in the FPD plane) of the FPD and to extract the sensitivity uniformly. In gain calibration, a gain correction map having in-plane distribution information acquired by imaging under a predetermined X-ray condition (the same X-ray condition as in the X-ray imaging) and in a state in which no subject is present is acquired. The in-plane distribution information is represented by a two-dimensional distribution on the detection surface of the data output from the FPD, and is also a two-dimensional distribution of sensitivity. Therefore, when collecting an X-ray image by the actual examination (X-ray imaging), an X-ray image with no unevenness is output by gain correction in which a gain correction map having in-plane distribution information acquired by gain calibration is applied.

However, depending on an FPD to be used, it has been found that a ring-like artifact as shown in FIG. 13A occurs in a high voltage image captured under a high voltage condition (high tube voltage X-ray condition). Further, depending on the FPD to be used, it has been found that unevenness as shown in FIG. 13B occurs in a low voltage image captured under a low voltage condition (low tube voltage X-ray condition). In particular, depending on a high voltage (140 kV) higher than the voltage used for chest imaging (80 kV to 100 kV) and an FPD to be used, a ring-like artifact tends to occur in the image (high voltage image), and in a subtraction image, a ring-like artifact as shown by the dotted line in FIG. 14A is emphasized. As the voltage applied to the X-ray tube becomes higher in this manner, a ring-like artifact may sometimes occur due to the individual differences in the FPD to be used. Such a ring-like artifact has been found to interfere with the quantitative measurement.

It is considered that the above-described phenomenon is caused by the non-uniformity of the film thickness of the X-ray conversion film (e.g., cesium iodide (CsI)). Findings have been acquired in which when an X-ray conversion film is formed by a proximity deposition method, the film thickness of the end portion is reduced and the above-described phenomenon tends to occur when the voltage becomes higher. From the above findings, it has been found that when gain correction is applied to a bone density measurement by a DXA method, the ring-like artifact can be eliminated in the subtraction image, which in turn can improve the accuracy of a bone density analysis.

Based on such findings, the present invention has the following configuration.

In other words, a bone density measuring apparatus according to the present invention is a bone density measuring apparatus including: an X-ray tube configured to emit X-rays; a detector configured to detect the X-rays emitted from the X-ray tube; a first gain correction map generation means configured to generate a first gain correction map having in-plane distribution information represented by a two-dimensional distribution at a detection surface of data output from the detector by detecting the X-rays emitted from the X-ray tube by the detector in a state in which no subject is present under a high tube voltage X-ray condition which is a high voltage condition in which a high voltage is applied to the X-ray tube; a second gain correction map generation means configured to generate a second gain correction map having in-plane distribution information represented by a two-dimensional distribution at a detection surface of data output from the detector by detecting the X-rays emitted from the X-ray tube by the detector in a state in which no subject is present under a low tube voltage X-ray condition which is a low voltage condition in which a low voltage lower than the high voltage is applied to the X-ray tube; a high voltage image generation means configured to generate a high voltage image captured by the detector by detecting the X-rays emitted from the X-ray tube and transmitted through the subject by the detector under the high tube voltage X-ray condition in which a high voltage of the same value as the high voltage applied to the X-ray tube at the time of generating the first gain correction map by the first gain correction map generation means is applied to the detector; a low voltage image generation means configured to generate a low voltage image captured by the detector by detecting the X-rays emitted from the X-ray tube and transmitted through the subject by the detector under the low tube voltage X-ray condition in which a low voltage of the same value as the low voltage applied to the X-ray tube at the time of generating the second gain correction map by the second gain correction map generation means is applied to the detector; a first gain correction means configured to perform a gain correction of the high voltage image generated by the high voltage image generation means using the first gain correction map generated by the first gain correction map generation means; a second gain correction means configured to perform a gain correction of the low voltage image generated by the low voltage image generation means using the second gain correction map generated by the second gain correction map generation means; and a subtraction processing means configured to perform a subtraction of the high voltage image after the gain correction by the first gain correction means and the low voltage image after the gain correction by the second gain correction means, wherein the bone density is measured by an image after subtraction processing by the subtraction processing means.

### Function and Effects

According to the bone density measuring apparatus of the present invention, by detecting the X-rays emitted from the X-ray tube by the detector under the high tube voltage X-ray condition which is a high voltage condition in which a high voltage is applied to the X-ray tube in a state in which no subject is present, a first gain correction map having in-plane distribution information represented by a two-dimensional distribution at the detection surface of the data output from the detector is generated. In the same manner, by detecting the X-rays emitted from the X-ray tube under the low tube voltage X-ray condition which is a low voltage condition in which a low voltage lower than the high voltage is applied to the X-ray tube in a state in which no subject is present, a second gain correction map having in-plane distribution information represented by a two-dimensional distribution at the detection plane of the data output from the detector is generated. The first gain correction map/the second gain correction maps is generated as a map having in-plane distribution information acquired by imaging under the high tube voltage X-ray condition/the low tube voltage X-ray condition in a state in which no subject is present.

Then, by detecting the X-rays emitted from the X-ray tube and transmitted through a subject by the detector under the high tube voltage X-ray condition in which a high voltage having the same value as the high voltage applied to the X-ray tube at the time of generating the first gain correction map is applied to the X-ray tube, a high voltage image captured by the detector is generated. In the same manner, by detecting the X-rays emitted from the X-ray tube and transmitted through a subject under the low tube voltage X-ray condition in which a low voltage equal to the low voltage applied to the X-ray tube at the time of generating the second gain correction map is applied to the X-ray tube, the low voltage image captured by the detector is generated. As described above, at the time of the bone density imaging, a high voltage and a low voltage having the same value at the time of the first gain correction map generation and the second gain correction map generation are applied to the X-ray tube to irradiate the subject with the X-rays emitted from the X-ray tube to generate a high voltage image and a low voltage image, respectively.

By performing the gain correction of the high voltage image using the first gain correction map and performing the gain correction of the low voltage image using the second gain correction map, it is possible to suppress unevenness and artifacts caused by unevenness in the high voltage image/the low voltage image after the gain correction under the high tube voltage X-ray condition/the low voltage image after the gain correction under the high tube voltage X-ray condition/the low tube voltage X-ray condition. Therefore, it is also possible to suppress unevenness and artifacts caused by unevenness in the image (subtraction image) after the subtraction processing acquired by subtracting the high voltage image after the gain correction and the low voltage image after the gain correction. As a result, by performing the gain correction of the high voltage image/the low voltage image using the first gain correction map/the second gain correction map having in-plane distribution information under the high tube voltage X-ray condition/the low tube voltage X-ray condition and performing the subtraction of the high voltage image/the low voltage image after the gain correction, the accuracy of the bone density analysis can be improved.

In the bone density measuring apparatus according to the present invention, the apparatus preferably further includes a reference gain correction map acquisition means configured to acquire a reference gain correction map having initially set (i.e., default) in-plane distribution information; a reference gain correction map storage means configured to store the reference gain correction map; and a usage condition switching means configured to switch a usage condition of a gain correction map described below. (a) A gain correction of an X-ray image acquired at the time of the normal X-ray imaging is performed using the reference gain correction map stored by the reference gain correction map storage means at the time of a normal X-ray imaging. On the other hand, (b) at a time of the bone density imaging by subtraction by the subtraction processing means, a gain correction of the high voltage image is performed using the first gain correction map having in-plane distribution information under the high tube voltage X-ray condition and a gain correction of the low voltage image is performed using the second gain correction map having in-plane distribution information under the low tube voltage X-ray condition. In the case of performing normal X-ray imaging other than bone density imaging, the in-plane distribution information under the same X-ray condition as that at the time of normal X-ray imaging is initially set, and the reference gain correction map having initially set in-plane distribution information is acquired and stored in the reference gain correction map storage means.

In summary, as described in (a), at the time of normal X-ray imaging, using the reference gain correction map stored by the reference gain correction map means, the gain correction of the X-ray image acquired at the time of normal X-ray imaging is performed, and as described in (b), at the time of bone density measuring, the usage condition of the gain correction map is switched so as to perform the gain correction of the high voltage image/the low voltage image using the first gain correction map/the second gain correction map having the in-plane distribution information under the high tube voltage condition/the low tube voltage condition. As described above, without using a dedicated bone density measuring apparatus, a common X-ray imaging apparatus can be used to perform normal X-ray imaging to which the gain correction is applied and bone density imaging to which the gain correction is applied.

As described above, in order to narrow down from the energy spectrum of X-rays to a specific energy component, it preferable to combine the high tube voltage X-ray condition (high voltage condition in which a high voltage is applied to the X-ray tube) and the tube voltage X-ray condition (low voltage condition in which a low voltage is applied to the X-ray tube) and two types of filters, i.e., a filter for a high voltage mode and a filter for a low voltage mode may be combined. That is, these filters are provided so as to be switchably arranged on the irradiation side of the X-ray tube. The first gain correction map is generated by detecting the X-rays emitted from the X-ray tube in a state in which the filter for a high voltage mode is arranged on the irradiation side of the X-ray tube under the high tube voltage X-ray condition in a state in which no subject is present. In the same manner, the second gain correction map is generated by detecting the X-rays emitted from the X-ray tube in a state in which the filter for a low voltage mode is arranged on the irradiation side of the X-ray tube under the low tube voltage X-ray condition in a state in which no subject is present. And, the high voltage image is generated by detecting the X-rays emitted from the X-ray tube and transmitted through the subject in a state in which the filter for a high voltage mode is arranged on the irradiation side of the X-ray tube under the high tube voltage X-ray condition. In the same manner, the low voltage image is generated by detecting the X-rays emitted from the X-ray tube and transmitted through the subject by the detector in a state in which the filter for the low voltage mode is arranged on the irradiation side of the X-ray tube under the low tube voltage X-ray condition.

In the bone density imaging, it is preferable to adopt an operative method called “slot imaging” in which a single X-ray image is generated by coupling a plurality of X-ray images captured by an irradiation field of the slit-like X-rays in a body axis direction of a subject. Specifically, the bone density measuring apparatus according to the present invention is provided with a collimator configured to form a slit-like irradiation field by limiting an irradiation region of the X-rays emitted from the X-ray tube and an irradiation field moving mechanism configured to relatively move the slit-like irradiation field in a body axis direction of the subject with respect to the detector by relatively moving the X-ray tube and the collimator in the body axis direction of the subject with respect to the detector.

The slit-like first gain correction maps each corresponding to the slit-like irradiation field are coupled in the body axis direction, wherein the slit-like first gain correction maps are generated by detecting the X-rays of the slit-like irradiation field emitted from the X-ray tube under the high tube voltage X-ray condition and formed by the collimator each time the irradiation field is moved by the irradiation field moving mechanism. By coupling them as described above, a single first gain correction map corresponding to the entire surface of the detector is generated. In the same manner, the slit-like second gain correction maps each corresponding to the slit-like irradiation field are coupled in the body axis direction, wherein the slit-like second gain correction maps are generated by detecting the X-rays of the slit-like irradiation field emitted from the X-ray tube under the low tube voltage X-ray condition and formed by the collimator in a state in which no subject is present each time the irradiation field is moved by the irradiation field moving mechanism. By coupling them as described above, a single second gain correction map corresponding to the entire surface of the detector is generated.

The slit-like high voltage images each corresponding to the slit-like irradiation field are coupled in the body axis direction, wherein the slit-like high voltage images are generated by detecting the X-rays of the slit-like irradiation field emitted from X-ray tube under the high tube voltage X-ray condition and formed by the collimator and transmitted through the subject each time the irradiation field is moved by the irradiation field moving mechanism. By coupling in this manner, a single high voltage image corresponding to the entire surface of the detector is generated. In the same manner, the slit-like high voltage images each corresponding to the slit-like irradiation field are coupled in the body axis direction, wherein the slit-like high voltage images are generated by detecting the X-rays of the slit-like irradiation field emitted from X-ray tube under the low tube voltage X-ray condition and formed by the collimator and transmitted through the subject each time the irradiation field is moved by the irradiation field moving mechanism. By coupling in this manner, a single low voltage image corresponding to the entire surface of the detector is generated.

In the case of emitting the X-rays on the entire surface of the detector without limiting the irradiation region of the X-rays, since the X-rays are incident on the end portion of the detector in an oblique direction, distortion occurs in the X-ray image at the end portion. On the other hand, in the case of the slot imaging, since the X-rays are incident perpendicularly on the detection surface of the detector, distortion of the X-ray image (first gain correction map/second gain correction map, high voltage image/low voltage image) can be suppressed. Further, in the case of the slot imaging, it is possible to acquire a high-quality X-ray image (first gain correction map/second gain correction map, high voltage image/low voltage image) in which the effect of scattered radiation is suppressed.

Further, the bone density measuring apparatus may be further provided with an imaging system moving mechanism configured to relatively move an imaging system composed of the X-ray tube and the detector in the body axis direction with respect to the subject and a long image generation mean configured to generate a long image by coupling images after subtraction processing (subtraction image) in the body axis direction, each of the images being generated each time the imaging system is moved by the imaging system moving mechanism. In the bone density imaging, the present invention may be applied to “long imaging” in which a long image (which is wider than the detecting area on the entire surface of the detector) is generated by coupling a plurality of X-ray images in the body axis direction in the same manner as in the slot imaging.

Further, the slot imaging and the long imaging may be combined. Specifically, in the slot imaging, slit-like X-ray images are respectively generated while moving the X-ray tube and the collimator in the body axis direction with respect to the detector, and a single X-ray image corresponding to the entire surface of the detector is generated by coupling these X-ray images in the body axis direction, and then a single X-ray image corresponding to the entire surface of the detector is generated by slot imaging while moving the imaging system in the body axis direction with respect to the subject. The above is repeated. Then, a long image is generated by coupling the generated X-ray images in the body axis direction. In the case of the present invention, a long subtraction image is generated as a long image by coupling the images respectively generated after the subtraction processing (subtraction image) in the body axis direction.

Note that it may be considered that the accuracy improvement can be expected if gain correction is performed for each pixel at the time of performing the gain correction, but the accuracy improvement cannot be actually expected. Due to the fluctuation of pixel values (i.e., statistical error), the actual pixel values are not true values. Therefore, if a high voltage image/a low voltage image is subjected to the gain correction for each pixel by using a gain correction map which is not a true value, there is a fear that the accuracy is rather lowered. Therefore, the bone density measuring apparatus may further include: a first gain correction map segment means configured to segment the first gain correction map into a plurality of regions; a second gain correction map segment means configured to segment the second gain correction map into a plurality of regions; a first gain correction map correction means configured to correct the first gain correction map by smoothing values of the in-plane distribution information in each region segmented by the first gain correction map segment means; and a second gain correction map correction means configured to correct the second gain correction map by smoothing values of the in-plane distribution information in each region segmented by the second gain correction map segment means. In this manner, by performing the gain correction of the high voltage image/the low voltage image using the first gain correction map/the second gain correction map corrected by smoothing the values of in-plane distribution information in each region, the gain correction can be performed appropriately and accurately. It should be noted that the shapes and the number of the regions which are segment targets are not required to be the same between the first gain correction map/the second gain correction map. An example of the smoothing includes acquiring an average value, but is not limited to acquiring the average value. For example, the smoothing may be performed using the median value or the smoothing may be performed using the mode value. In other words, the smoothing may be performed using statistics.

Further, the bone density imaging method according to the present invention is a bone density measuring method for measuring a bone density using a bone density measuring apparatus equipped with an X-ray tube configured to emit X-rays and a detector configured to detect the X-rays emitted from the X-ray tube; the bone density measuring method comprising:

a first gain correction map generation step for generating a first gain correction map having in-plane distribution information represented by a two-dimensional distribution at a detection surface of data output from the detector by detecting the X-rays emitted from the X-ray tube by the detector in a state in which no subject is present under a high tube voltage X-ray condition which is a high voltage condition in which a high voltage is applied to the X-ray tube;

a second gain correction map generation step for generating a second gain correction map having in-plane distribution information represented by a two-dimensional distribution at a detection surface of data output from the detector by detecting the X-rays emitted from the X-ray tube by the detector in a state in which no subject is present under a low tube voltage X-ray condition which is a low voltage condition in which a low voltage lower than the high voltage is applied to the X-ray tube;

a high voltage image generation step for generating a high voltage image captured by the detector by detecting the X-rays emitted from the X-ray tube and transmitted through the subject by the detector under the high tube voltage X-ray condition in which a high voltage of the same value as the high voltage applied to the X-ray tube at the time of generating the first gain correction map in the first gain correction map generation step is applied to the detector;

a low voltage image generation step for generating a low voltage image captured by the detector by detecting the X-rays emitted from the X-ray tube and transmitted through the subject by the detector under the low tube voltage X-ray condition in which a low voltage of the same value as the low voltage applied to the X-ray tube at the time of generating the second gain correction map by the second gain correction map generation means is applied to the detector;

a first gain correction step for performing a gain correction of the high voltage image generated in the high voltage image generation step using the first gain correction map generated in the first gain correction map generation step;

a second gain correction step for performing a gain correction of the low voltage image generated in the low voltage image generation step using the second gain correction map generated in the second gain correction map generation step; and

a subtraction processing step for performing a subtraction of the high voltage image after the gain correction in the first gain correction step and the low voltage image after the gain correction in the second gain correction step, wherein the bone density is measured by an image after subtraction processing in the subtraction processing step.

### Effects

According to the bone density imaging method according to the present invention, the bone density imaging can be suitably performed by performing each step (the first gain correction map generation step, the second gain correction map generation step, the high voltage image generation step, the low voltage image generation step, the first gain correction step, the second gain correction step, and the subtraction processing step), and the accuracy of the bone density analysis can be improved.

In the bone density imaging method according to the present invention, at the time of the slot imaging in which a single X-ray image is generated by coupling a plurality of X-ray images captured by a slit-like X-ray irradiation field in the body axis direction of the subject, a slit-like irradiation field is formed by limiting the irradiation region of the X-rays emitted from the X-ray tube with the collimator, and the slit-like irradiation field is relatively moved in the body axis direction with respect to the detector by relatively moving the X-ray tube and the collimator in the body axis direction with respect to the detector to perform the slot imaging.

In the first gain correction map generation step, the slit-like first gain correction maps each corresponding to the slit-like irradiation field are coupled in the body axis direction, wherein the slit-like first gain correction maps are formed by detecting the X-rays of the slit-like irradiation field emitted from the X-ray tube and formed by the collimator under the high tube voltage X-ray condition in a state in which no subject is present each time the slit-like irradiation field is relatively moved in the body axis direction with respect to the detector, and a single first gain correction map corresponding to an entire surface of the detector is generated by coupling the slit-like first gain correction maps in the body axis direction. In the same manner, in the second gain correction map generation step, the slit-like second gain correction maps each corresponding to the slit-like irradiation field are coupled in the body axis direction, wherein the slit-second gain correction maps are formed by detecting the X-rays of the slit-like irradiation field emitted from the X-ray tube and formed by the collimator under the low tube voltage X-ray condition in a state in which no subject is present each time the slit-like irradiation field is relatively moved in the body axis direction with respect to the detector, and a single second gain correction map corresponding to an entire surface of the detector is generated by coupling the slit-like second gain correction maps in the body axis direction.

In the case of adopting such slot imaging, the following modes can be conceivable.

That is, the second gain correction map generation step is performed after the first gain correction map generation step. Alternatively, the first gain correction map generation step is performed after the second gain correction map generation step. As described above, the first gain correction map generation step and the second gain correction map generation step may be performed separately in time.

In contrast, the slit-like first gain correction map and the slit-like second gain correction map are alternately generated each time the slit-like irradiation field is relatively moved in the body axis direction with respect to the detector by relatively moving the slit-like irradiation field in the body axis direction with respect to the detector while alternately applying the high voltage and the low voltage to the X-ray tube. The first gain correction map generation step for generating a single slit-like first gain correction map corresponding to the entire surface of the detector by coupling the slit-like gain correction maps in the body axis direction and the second gain correction map generation step for generating a single slit-like second gain correction map corresponding to the entire surface of the detector by coupling the slit-like gain maps in the body axis direction may be performed simultaneously. As described above, the first gain correction map generation step and the second gain correction map generation step may be performed by relatively moving the slit-like irradiation field in the body axis direction with respect to the detector while alternately applying the high voltage and the low voltage to the X-ray tube.

Further, in the bone density imaging method according to the present invention, the high voltage image generation step for generating the high voltage image and the low voltage image generation step for generating the low voltage image is preferably performed simultaneously by alternately applying a high voltage having the same value as that at the time of generating the first gain correction map and a low voltage having the same value as that at the time of generating the second gain correction map to the X-ray tube to emit the X-rays from the X-ray tube to the subject. This makes it possible to simultaneously acquire the high voltage image and the low voltage image in the single imaging including slot imaging and long imaging.

### Effects of the Invention

According to the bone density measuring apparatus and the bone density imaging method according to the present invention, the first gain correction map/the second gain correction map having in-plane distribution information represented by a two-dimensional distribution at the detection surface of the data output from the detector is generated by detecting the X-rays emitted from the X-ray tube by the detector in a state in which no subject is present under the high tube voltage X-ray condition/the low tube voltage X-ray condition which is a high voltage condition in which a high voltage is applied to the X-ray tube/a low voltage condition in which a low voltage is applied to the X-ray tube. And the high voltage image/the low voltage image captured by the detector is generated by detecting the X-rays emitted from the X-ray tube under the high tube voltage X-ray condition/the low tube voltage X-ray condition in which the high voltage/the low voltage having the same value as that the high voltage/the low voltage applied to the X-ray tube at the time of generating the first gain correction map/the second gain correction map and transmitted through the subject. By performing the gain correction of the high voltage image using the first gain correction map and performing the gain correction of the low voltage image using the second gain correction map, it is possible to suppress unevenness and artifacts due to unevenness in the high voltage image/the low voltage image after the gain correction under the high tube voltage X-ray condition and the low tube voltage X-ray condition. Therefore, it is also possible to suppress unevenness and artifacts due to unevenness in the image after the subtraction processing (subtraction image) acquired by subtracting the high voltage image after the gain correction and the low voltage image after the gain correction. As a result, the accuracy of the bone density analysis can be improved.

### BRIEF DESCRIPTION OF THE DRAWINGS

FIG. 1 is a block diagram of a bone density measuring apparatus according to an embodiment.

FIG. 2 is a specific perspective view of a filter.

FIG. 3 is a perspective view of collimator leaves of a collimator.

FIG. 4 is a block diagram of an image processing unit.

FIG. 5 is a flowchart of a series of a bone density imaging method according to this embodiment.

FIG. 6 is an explanatory view showing a moving operation of an X-ray tube and a collimator.

FIG. 7 is an explanatory diagram showing the coupling of the slit-like first gain correction map/the slit-like second gain correction maps using a maximum-value projection method (MIP).

FIG. 8 shows an example of an embodiment related to a region segment of a first gain correction map.

FIG. 9 shows an example of an embodiment related to a region segment of a second gain correction map.

FIG. 10 is a schematic diagram of a bone and a soft tissue for explaining the principles of a DXA-method.

FIGS. 11A and 11B are schematic diagrams of a profile curve of a measurement site with low energy and high energy for explaining the measurement principle of the DXA method.

FIGS. 12A and 12B are schematic diagrams for explaining the calculation of a bone mineral quantitative analysis (bone mineral content) in the DXA method.

FIG. 13A shows an in-plane distribution under a high tube voltage X-ray condition, and FIG. 13B shows an in-plane distribution under a low tube voltage X-ray condition.

FIGS. 14A and 14B are subtraction images acquired by imaging a phantom simulating a bone, wherein FIG. 14A is a subtraction image when the gain correction of the present invention has not been performed and FIG. 14B is a subtraction image when the gain correction of the present invention has been performed.

### DESCRIPTION OF THE EMBODIMENTS

Hereinafter, embodiments of the present invention will be described with reference to the attached drawings.

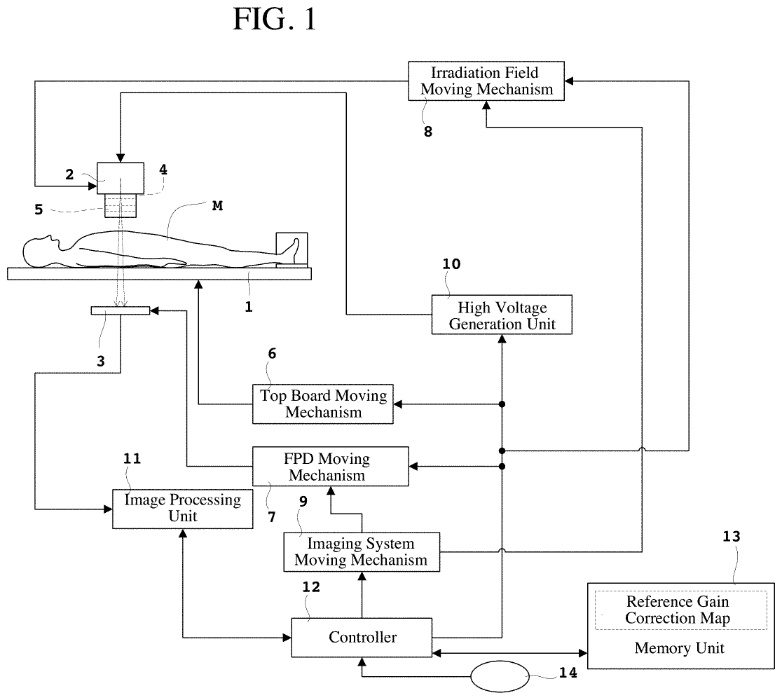

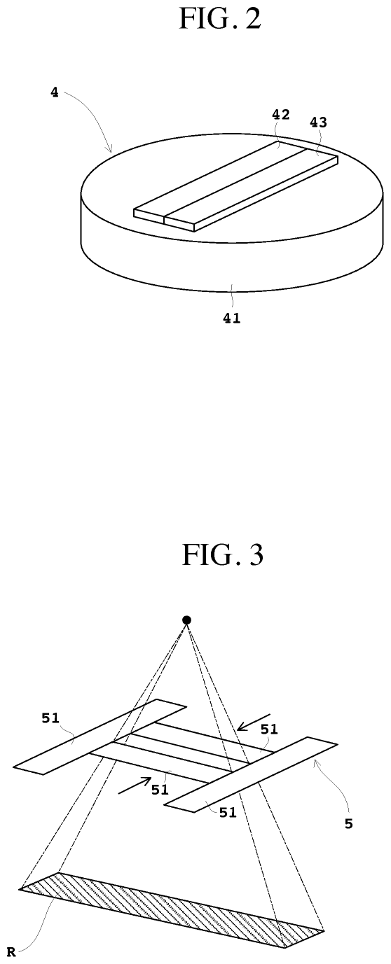

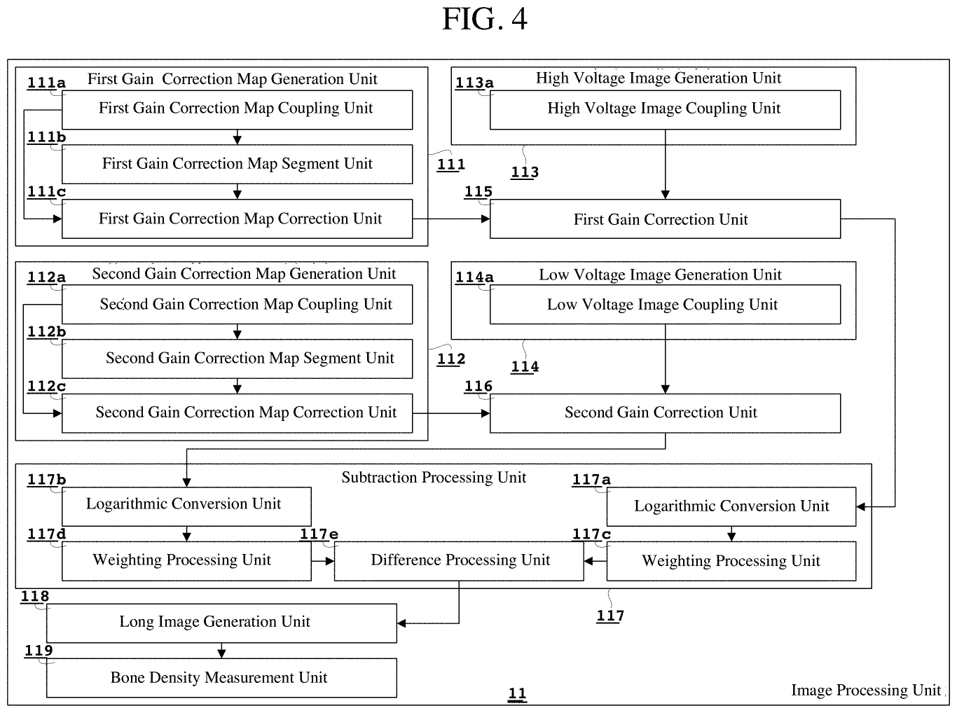

FIG. 1 is a block diagram of a bone density measuring apparatus according to an embodiment. FIG. 2 is a specific perspective view of a filter. FIG. 3 is a perspective view of collimator leaves of a collimator. FIG. 4 is a block diagram of an image processing unit. In this embodiment, a common X-ray imaging apparatus is used as the bone density measuring apparatus.

As shown in FIG. 1, the bone density measuring apparatus is provided with a top board 1 for placing a subject M thereon, an X-ray tube 2 for emitting X-rays, and a flat panel X-ray detector (FPD) 3 for detecting the X-rays emitted from the X-ray tube 2. The bone density measuring apparatus is further provided with two types of filters 4, i.e., a filter for a high voltage mode and a filter for a low voltage mode (metal filters 41 and 42 (see FIG. 2)), provided in such manner as to be switchably arranged on the irradiation side of the X-ray tube 2. Thus, an X-ray beam having two different energy peaks are emitted while switching the metal filter 41 for a high voltage mode and the low voltage mode metal filter 42. For slot imaging, a collimator 5 for forming a slit-like irradiation field by limiting the irradiation region R (see FIG. 3) of the X-rays emitted from the X-ray tube 2 is provided.

Note that the flat panel X-ray detector (FPD) 3 corresponds to the detector in the present invention.

The bone density measuring apparatus further includes: in addition to the above, a top board moving mechanism 6 for moving the top board 1 upward and downward and horizontally in the horizontal direction (in particular, in the body axis direction of the subject M which is a longitudinal direction); an FPD moving mechanism 7 for moving the FPD 3 horizontally in the horizontal direction (in particular, in the body axis direction of the subject M which is a longitudinal direction); an irradiation field moving mechanism 8 for moving the slit-like irradiation field in the body axis direction (longitudinal direction) by moving the X-ray tube 2 and the collimator 5 in the body axis direction (longitudinal direction) of the subject M; an imaging system moving mechanism 9 for moving an imaging system composed of the X-ray tube and the FPD 3 in the body axis direction (longitudinal direction); a high voltage generation unit 10 for generating a tube voltage and a tube current of the X-ray tube 2; an image processing unit 11 for performing image processing such as subtraction processing; a controller 12 for integrally performing control of these constituent units; a memory unit 13 for storing various gain correction maps (in particular, a reference gain correction map) and the like; and an input unit 14 for performing input by an operator.

Note that the controller 12 corresponds to the reference gain correction map acquisition means in the present invention. The memory unit 13 corresponds to the reference gain correction map storage means in the present invention. The input unit 14 corresponds to the usage condition switching means in the present invention.

The top board moving mechanism 6 is constituted by a linear rack in the vertical direction, a linear rack in the body axis direction (longitudinal direction), a pinion, a motor, an encoder (not shown), etc. The FPD moving mechanism 7 is constituted by a linear rack in the body axis direction (longitudinal direction), a pinion, a motor, an encoder (not shown), etc. The irradiation field moving mechanism 8 is constituted by a linear rack in the body axis direction (longitudinal direction), a pinion, a motor, an encoder (not shown), etc. Further, the imaging system moving mechanism 9 is configured so that the FPD moving mechanism 7 and the irradiation field moving mechanism 8 are driven synchronously in order to move the X-ray tube 2 and the FPD 3 synchronously in the same body axis direction (longitudinal direction) when performing the long imaging.

The high voltage generation unit 10 generates a tube voltage and a tube current for emitting X-rays and applies them to the X-ray tube 2. In the normal X-ray imaging, a voltage of about 80 kV to 100 kV is applied to the X-ray tube 2. In the bone density imaging of this embodiment, a high voltage of about 140 kV is applied to the X-ray tube 2 under the high tube voltage X-ray condition, and a low voltage of about 100 kV is applied to the X-ray tube 2 under the low tube voltage X-ray condition. As described above, in the bone density imaging, the high tube voltage and the low tube voltage are alternately switched by alternately applying the high voltage and the low voltage to the X-ray tube 2. In synchronization with the X-ray irradiation (exposure) from the X-ray tube 2 in accordance with the switching of the high tube voltage and the low tube voltage, the metal filter 41 for a high voltage mode and the metal filter 42 for a low voltage mode (see FIG. 2) is alternately switched.

In the bone density imaging of this embodiment, in order to cope with the slot imaging, it is enough to secure the metallic area in the filter 4 only by a slit. Therefore, as shown in FIG. 2, the filter 4 is configured by arranging two types of strip-like metal filters 41 and 42 on a normal filter base 43 for an entire surface. The high-speed switching of the metal filters 41 and 42 is realized in synchronization with the exposure signals under the control of the collimator 5 (see FIG. 1).

As shown in FIG. 3, the collimator 5 is provided with four collimator leaves 51. By moving the collimator leaves 51 in the directions of the arrows in FIG. 3, the size of the opening surrounded by the four collimator leaves 51 is adjusted. The irradiation region R of the X-rays passing through the opening is limited as shown in FIG. 3 by adjusting the opening into a slit-like shape. By controlling the irradiation region R of the X-rays in this manner, a slit-like irradiation region R is formed. Note that the black circle in FIG. 3 represents the focal point of the X-ray tube 2 (see FIG. 1).

Referring back to the explanation of FIG. 1, the controller 12 is composed of a central processing unit (CPU), etc., and the memory unit 13 is composed of a storage medium, such as, e.g., a ROM (Read-Only Memory) and a RAM (Random-Access Memory). The input unit 14 is composed of a pointing device, such as, e.g., a mouse, a keyboard, a joystick, a trackball, and a touch panel.

In this embodiment, the controller 12 acquires a reference gain correction map having initially set (default) in-plane distribution information. As described above, in this embodiment, a common X-ray imaging apparatus is used as the bone density measuring apparatus. Therefore, the FPD 3 detects the X-rays emitted from the X-ray tube 2 in a state in which no subject is present under the X-ray condition in which a voltage of about 80 kV to about 100 kV which is used for normal X-ray imaging is applied to the X-ray tube 2 when the device is installed. With this, the in-plane distribution information represented by a two-dimensional distribution at the detection surface of the data output by the FPD 3 is set as the initial setting, and a reference gain correction map having the initially set (default) in-plane distribution information of an initial setting is acquired. The acquired reference gain correction map is written to the memory unit 13 and stored.

Note that since the reference gain correction map may sometimes change with the secular change of the characteristics of the FPD 3, it is preferable to periodically acquire a reference gain correction map and periodically write and store it in the memory unit 13.

At the time of the normal X-ray imaging, the reference gain correction map stored in the memory unit 13 is read. The reference gain correction map is used to perform the gain correction of an X-ray image acquired at the time of the normal X-ray imaging. On the other hand, at the time of the bone density imaging, in order to switch the usage condition of the gain correction map, the operator inputs a command for switching the usage condition of the gain correction map to the input unit 14. With this, a high voltage image/a low voltage image is subjected to the gain correction by using a first gain correction map/a second gain correction map, which will be described later, without using the reference gain correction map stored in the memory unit 13.

Note that as long as the value of the high voltage/the low voltage used for the bone density imaging does not change, it is not necessary to acquire the first gain correction map/the second gain correction map each time the gain calibration is performed, and it is sufficient to perform the gain correction using a previously acquired first gain correction map/second gain correction map. However, in the same manner as in the reference gain correction map, since the first gain correction map/the second gain correction map may change with the secular change of the characteristics of the FPD 3, it is preferable to periodically acquire the first gain correction map/the second gain correction map.

The image processing unit 11 is constituted by a GPU (Graphics Processing Unit), etc. As shown in FIG. 4, the image processing unit 11 is provided with a first gain correction map generation unit 111, a second gain correction map generation unit 112, a high voltage image generation unit 113, a low voltage image generation unit 114, a first gain correction unit 115, a second gain correction unit 116, a subtraction processing unit 117, a long image generation unit 118, and a bone density measurement unit 119.

The first gain correction map generation unit 111 corresponds to the first gain correction map generation means in the present invention. The second gain correction map generation unit 112 corresponds to the second gain correction map generation means in the present invention. The high voltage image generation unit 113 corresponds to the high voltage image generation means in the present invention. The low voltage image generation unit 114 corresponds to the low voltage image generation means in the present invention. The first gain correction unit 115 corresponds to the first gain correction means in the present invention. The second gain correction unit 116 corresponds to the second gain correction means in the present invention. The subtraction processing unit 117 corresponds to the subtraction processing means in the present invention. The long image generation unit 118 corresponds to the long image generation mean in the present invention.

The first gain correction map generation unit 111 generates a first gain correction map having in-plane distribution information represented by a two-dimensional distribution at the detection surface of the data output from the FPD 3 by detecting the X-rays emitted from the X-ray tube 2 under the high tube voltage X-ray condition which is a high voltage condition in which a high voltage (140 kV) is applied to the X-ray tube 2 (see FIG. 1) in a state in which no subject is present.

In this embodiment, the first gain correction map generation unit 111 is equipped with a first gain correction map coupling unit 111a. The first gain correction map coupling unit 111a couples the slit-like first gain correction maps each corresponding to the slit-like irradiation field in the body axis direction (longitudinal direction). The slit-like first gain correction map is generated by detecting the X-rays of the slit-like irradiation field emitted from the X-ray tube 2 by the FPD 3 under the high tube voltage X-ray condition and formed by the collimator 5 (see FIG. 1) in a state in which no subject is present each time the irradiation field is moved by the irradiation field moving mechanism 8 (see FIG. 1). A single first gain correction map corresponding to the entire surface of the FPD 3 is generated by coupling the slit-like first gain correction maps with the first gain correction map coupling unit 111a.

Note that the first gain correction map coupling unit 111a corresponds to the first gain correction map coupling means in the present invention.

The first gain correction map generation unit 111 is further provided with a first gain correction map segment unit 111b and a first gain correction map correction unit 111c. The first gain correction map segment unit 111b segments the first gain correction map into a plurality of regions. The first gain correction map correction unit 111c corrects the first gain correction map by smoothing the values of the in-plane distribution information in each of the regions segmented by the first gain correction map segment unit 111b. The first gain correction map segment unit 111b may be configured such that an operator manually performs input setting, or the first gain correction map segment unit 111b may be configured by the input unit 14 (see FIG. 1).

Note that the first gain correction map segment unit 111b corresponds to the first gain correction map segment means in the present invention. Further note that the first gain correction map correction unit 111c corresponds to the first gain correction map correction means in the present invention.

The second gain correction map generation unit 112 generates a second gain correction map having in-plane distribution information represented by a two-dimensional distribution at the detection plane of the data output from the FPD 3 by detecting the X-rays emitted from X-ray tube 2 under the low tube voltage X-ray condition which is a low voltage condition in which a low voltage (100 kV) lower than the high voltage is applied to the X-ray tube 2 in a state in which no subject is present with the FPD.

In this embodiment, the second gain correction map generation unit 112 is provided with a second gain correction map coupling unit 112a. The second gain correction map coupling unit 112a couples the slit-like second gain correction maps each corresponding to the slit-like irradiation field in the body axis direction (longitudinal direction). The slit-like second gain correction maps are generated by detecting the X-rays of the slit-like irradiation field with the FPD 3 in a state in which no subject is present each time the slit-like second gain correction map is moved by the irradiation field moving mechanism 8, wherein the X-rays are emitted from the X-ray tube 2 under the low tube voltage X-ray condition and the slit-like irradiation field is formed by the collimator 5. A single second gain correction map corresponding to the entire surface of the FPD 3 is generated by coupling the slit-like second gain correction maps with the second gain correction map coupling unit 112a.

Note that the second gain correction map coupling unit 112a corresponds to the second gain correction map coupling means in the present invention.

The second gain correction map generation unit 112 is further provided with a second gain correction map segment unit 112b and a second gain correction map correction unit 112c. The second gain correction map segment unit 112b segments the second gain correction map into a plurality of regions. The second gain correction map correction unit 112c corrects the second gain correction map by smoothing the values of the in-plane distribution information in each of the regions segmented by the second gain correction map segment unit 112b. Similar to the first gain correction map segment unit 111b, the second gain correction map segment unit 112b may be configured such that the operation manually perform input setting, or the second gain correction map segment unit 112b may be configured by the input unit 14.

Note that the second gain correction map segment unit 112b corresponds to the second gain correction map segment means in the present invention, and the second gain correction map correction unit 112c corresponds to the second gain correction map correction means in the present invention.

The high voltage image generation unit 113 generates a high voltage image captured by the FPD 3 by detecting the X-rays emitted from the X-ray tube 2 under a high tube voltage X-ray condition in which a high voltage (140 kV) having the same value as the high voltage applied to the X-ray tube 2 at the time of generation of the first gain correction map by the first gain correction map generation unit 111 and transmitted through a subject M (see FIG. 1) by the FDP 3.

In this embodiment, the high voltage image generation unit 113 is equipped with a high voltage image coupling unit 113a. The high voltage image coupling unit 113a couples the slit-like high voltage images each corresponding to the slit-like irradiation field in the body axis direction (longitudinal direction) of the subject M. The slit-like high voltage images are generated by detecting the X-rays of the slit-like irradiation field each time the slit-like irradiation field is moved by the irradiation field moving mechanism 8, and the X-rays are emitted from the X-ray tube 2 under the high tube voltage X-ray condition and transmitted through the subject M and the slit-like field is formed by the collimator 5. A single high voltage image corresponding to the entire surface of the FPD 3 is generated by coupling the slit-like high voltage images with the high voltage image coupling unit 113a.

Note that the high voltage image coupling unit 113a corresponds to the high voltage image coupling unit in the present invention.

The low voltage image generation unit 114 generates a low voltage image captured by the FPD 3 by detecting the X-rays emitted from the X-ray tube 2 and transmitted through the subject M under a low tube voltage X-ray condition in which a low voltage (100 kV) of the same value as the low voltage applied to the X-ray tube 2 at the time of generation of the second gain correction map by the second gain correction map generation unit 112 is applied to the X-ray tube 2 with the FPD 3.

In this embodiment, the low voltage image generation unit 114 is equipped with a low voltage image coupling unit 114a. The low voltage image coupling unit 114a couples the slit-like low voltage images each corresponding to the slit-like irradiation field in the body axis direction (longitudinal direction) generated by detecting the X-rays of the slit-like irradiation fields emitted from the X-ray tube 2 under the low tube voltage X-ray condition and formed by the collimator 5 and transmitted through the subject M each time the slit-like irradiation field is moved by the irradiation field moving mechanism 8. A single low voltage image corresponding to the entire surface of the FPD 3 is generated by coupling the slit-like low voltage images with the low voltage image coupling unit 114a.

Note that the low voltage image coupling unit 114a corresponds to the low voltage image coupling means in the present invention.

The first gain correction unit 115 performs the gain correction of the high voltage image generated by the high voltage image generation unit 113 by using the first gain correction map generated by the first gain correction map generation unit 111. The second gain correction unit 116 performs the gain correction of the low voltage image generated by the low voltage image generation unit 114 by using the second gain correction map generated by the second gain correction map generation unit 112.

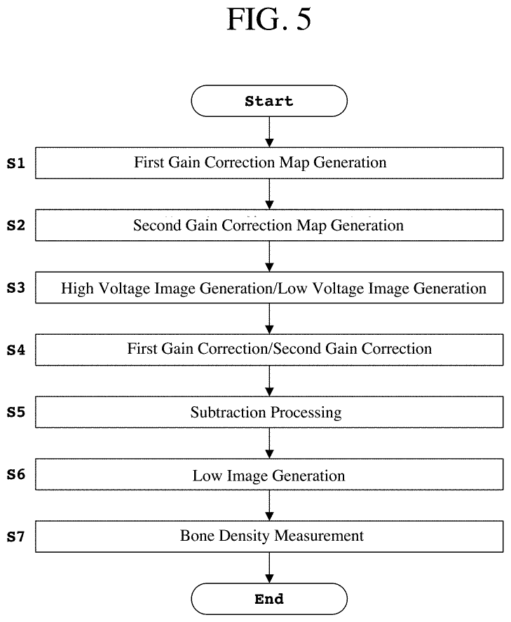

The subtraction processing unit 117 performs the subtraction of the high voltage image after the gain correction by the first gain correction unit 115 and the low voltage image after the gain correction by the second gain correction unit 116. The subtraction processing unit 117 is provided with logarithmic conversion units 117a and 117b, weighting processing units 117c and 117d, and a difference processing unit 117e. The specific method for acquiring the image (subtraction image) after the subtraction processing will be described later with reference to FIG. 5 to FIG. 12B.

The long image generation unit 118 generates a long image (long subtraction image) by coupling the images (subtraction images) after the subtraction processing by the subtraction processing unit 117 in the body axis direction (longitudinal direction), wherein the images are generated each time the imaging system is moved by the imaging system moving mechanism 9 (see FIG. 1)

The bone density measurement unit 119 performs the measurement of the bone density using the long image (long subtraction image). The specific method of acquiring the bone density will be described later with reference to FIG. 5 to FIG. 12B.

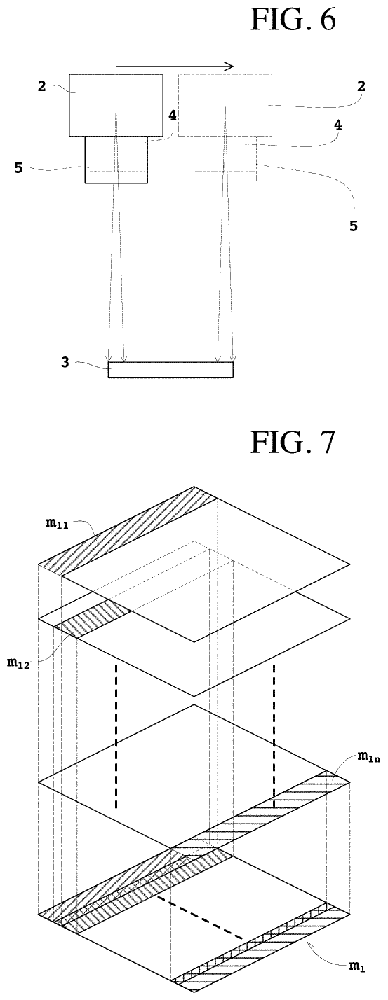

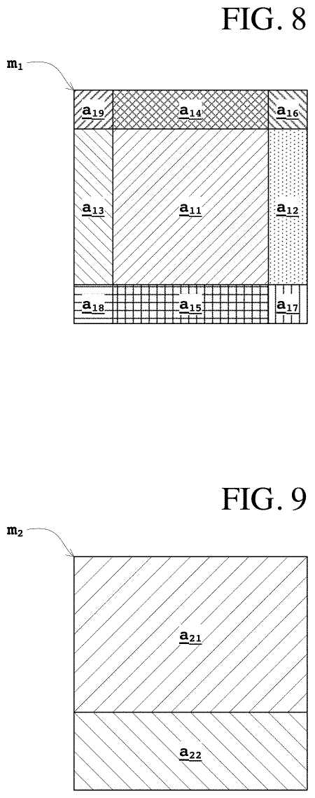

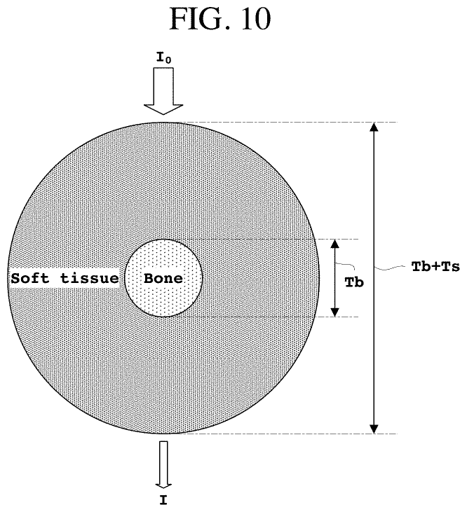

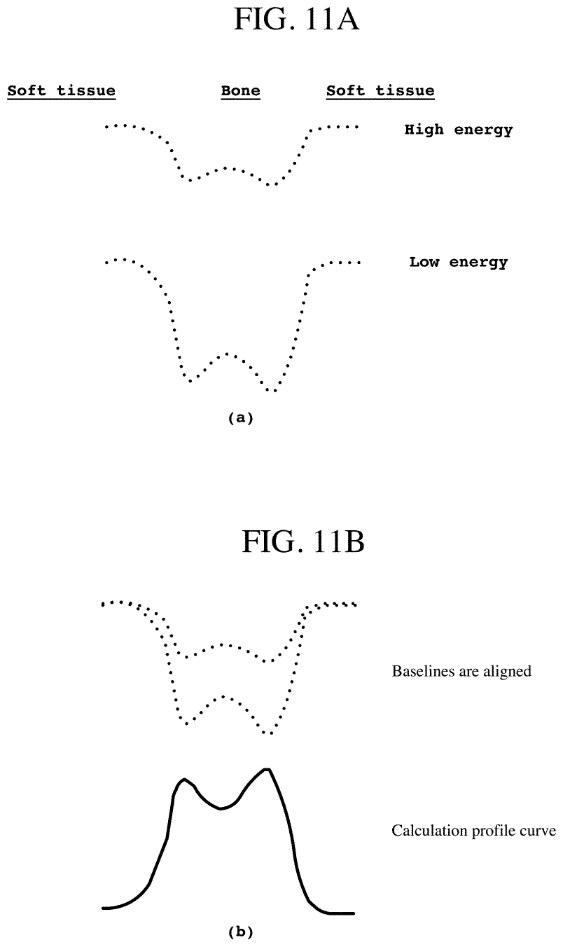

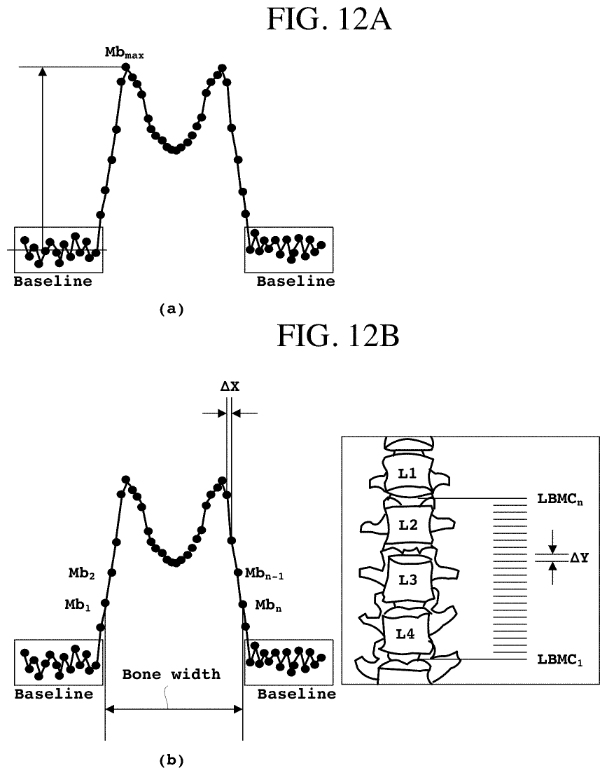

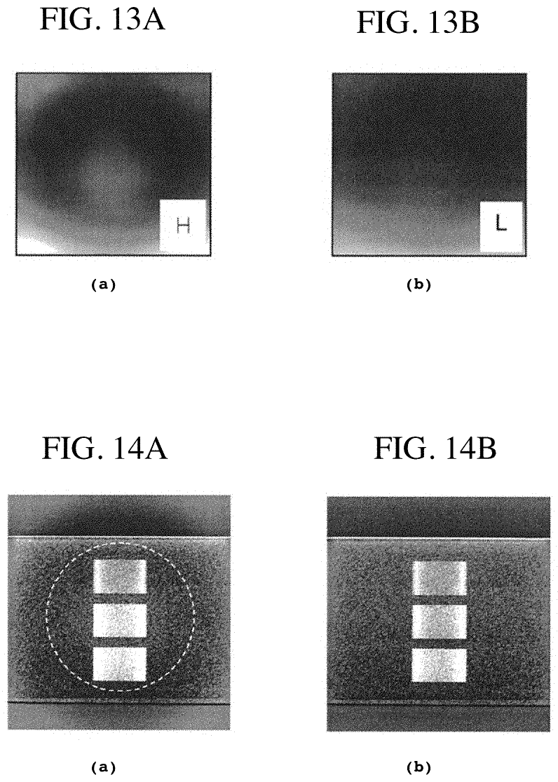

Next, a specific bone density imaging method will be described with reference to FIG. 5 to FIG. 12B. FIG. 5 is a flowchart of a series of the bone density imaging method according to this embodiment. FIG. 6 is an explanatory view showing the moving operation of the X-ray tube and the collimator. FIG. 7 is an explanatory view showing the coupling of the slit-like first gain correction maps/the slit-like second gain correction maps using a maximum-value projection method. FIG. 8 is an example of an embodiment related to a region segment of the first gain correction map. FIG. 9 is an example of an embodiment related to a region segment of the second gain correction map. FIG. 10 is a schematic diagram of a bone and a soft tissue for explaining the principle of a DXA-method. FIGS. 11A and 11B are schematic diagrams of a profile curve of a measurement site with low energy and high energy for explaining the measurement principle of the DXA method. FIGS. 12A and 12B are schematic diagrams for explaining the calculation of a bone mineral quantitative analysis (bone mineral content) in the DXA method.

As described above, it is unnecessary to acquire the first gain correction map/the second gain correction map each time the gain calibration is performed, and it is unnecessary to perform Step S1 and Step S2 of FIG. 5 each time. However, as described above, since the characteristics of the FPD 3 (see FIG. 1) may change over time, and therefore there is a possibility that the first gain correction map/the second gain correction map changes. For this reason, Step S1 and Step S2 are performed periodically.

### (Step S1) First Gain Correction Map Generation

First, without placing a subject M (see FIG. 1) on the top board 1 (see FIG. 1), the X-ray tube 2 and the collimator 5 are moved in the body axis direction (longitudinal direction) of the subject M in a state in which the FPD 3 is fixed as shown in FIG. 6. The moving distance at this time may be a distance to the extent that no gap occurs between the adjacent slit-like first gain correction map gaps, and may be equal to or less than the slit width of the first gain correction map. Ideally, when the slit width of the first gain correction map is defined as the moving distance, they do not overlap in the adjacent slit-like first gain correction map. However, in the first gain correction map coupling unit 111a (see FIG. 4), the slit-like first gain correction maps are coupled by using a maximum-value projection method (MIP: Maximum Intensity Projection). Therefore, the adjacent slit-like first gain correction maps may overlap with each other. The reason will be described later with reference to FIG. 7.

By relatively moving the X-ray tube 2 and the collimator 5 with respect to the FPD 3 in the body axis direction (longitudinal direction), the slit-like irradiation field is relatively moved with respect to the FPD 3 in the body axis direction (longitudinal direction). For this purpose, in this embodiment, the irradiation field moving mechanism 8 (see FIG. 1) moves the X-ray tube 2 and the collimator 5 in the body axis direction (longitudinal direction) in a state in which the FPD 3 is fixed. By this moving, the slit-like irradiation field is moved in the body axis direction (longitudinal direction) in a state in which the FPD 3 is fixed.

Each time the slit-like irradiation field is moved by the irradiation field moving mechanism 8, the X-ray tube 2 emits X-rays under the high tube voltage X-ray condition, and the FPD 3 detects the X-rays of the slit-like irradiation field formed by the collimator 5 in a state in which no subject is present. Note that the metal filter is switched to the metal filter 41 for a high voltage mode in synchronization with the exposure signal under the high tube voltage X-ray condition (see FIG. 2). This high voltage mode metal filter 41 makes it possible to narrow down the energy spectrum of the X-rays to a certain energy component.

The data output from the FPD 3 in a state in which no subject is present is also a two-dimensional distribution of the sensitivity of the FPD 3 as described in the findings described in the section of “Means for Solving the Problems”. Therefore, the two-dimensional distribution of the sensitivity is generated as a first gain correction map having in-plane distribution information represented by a two-dimensional distribution at the detection surface of the data output from the FPD 3. This first gain correction map has a shape corresponding to the slit-like irradiation field, and what is required is a single map corresponding to the entire surface of the FPD 3. Thus, each time the slit-like irradiation field is moved by the irradiation field moving mechanism 8, slit-like first gain correction maps each corresponding to the slit-like irradiation field are generated, and the first gain correction map coupling unit 111a couples the slit-like first gain correction maps in the longitudinal direction. By coupling in this manner, a single first gain correction map corresponding to the entire surface of the FPD 3 is generated.

Using a maximum-projection method (MIP), the slit-like first gain correction maps m11, m12, . . . , m1n, are coupled in the body axis direction (longitudinal direction) as shown in FIG. 7. When the respective maps are coupled in cases where the adjacent slit-like first gain correction maps overlap, the coupling of the respective maps is performed by weighted addition according to distances of the center positions of the overlapped maps. However, the distance and the coefficient of weighting (weighting coefficient) must be sequentially acquired, which takes time and labor in the coupling processing.

Therefore, each of the slit-like first gain correction maps m11, m12, . . . , m1n is considered as a circular cross-section (coronal cross-section) parallel to the horizontal plane of the subject M as shown in FIG. 7, the maps m11, m12, . . . , m1n are coupled by considering the maximum value of the pixel value in the projection path (see one-dot chain line in FIG. 7) by the maximum value projection method (MIP) as a projection image to generate a single first gain correction map m1 corresponding to the entire surface of the FPD 3. In summary, the slit-like first gain correction maps m11, m12, . . . , m1n are coupled in the body axis direction (longitudinal direction) using a maximum-projection method (MIP) to generate a first gain correction map m1 corresponding to the entire surface of the FPD 3.

From the above, even if the adjacent slit-like first gain correction maps overlap, the pixel having a higher pixel value in the overlapping region is considered to be the pixel of a single first gain correction map m1 corresponding to the entire surface of the FPD 3. Therefore, the weighting factor corresponding the distance of the center positions of the overlapped regions and the weighting factor as required in a prior art are not required, so that it is possible to simply generate a single first gain correction map m1 corresponding to the entire surface of the FPD 3.

In this embodiment, the slit-like first gain correction maps are coupled by using a maximum value projection method (MIP), but the present invention not limited to use a maximum value projection method (MIP). The slit-like first gain correction maps may be coupled by weight addition according to the distance of the center positions of the overlapped maps as in a prior art.

The in-plane distribution information generated as described above in the first gain correction map is represented by a two-dimensional distribution of the sensitivity of the FPD 3 as described above. On the other hand, the in-plane distribution information in the first gain correction map, including a second gain correction map which will be described later, includes fluctuations (statistical errors). Therefore, when gain corrections, which will be described later, by the first gain correction unit 115 and the second gain correction unit 116 shown in FIG. 4 are performed for each pixel, the accuracy may be rather deteriorated.

Therefore, the first gain correction map segment unit 111b (see FIG. 4) segments the first gain correction map m1 into a plurality of regions (for example, nine regions a1, a12, a13, a14, a15, a16, a17, a18, and a19 in FIG. 8). The first gain correction map correction unit 111c (see FIG. 4) corrects the first gain correction map m1 by smoothing the values of the in-plane distribution information in the respective regions a11, a12, a13, a14, a15, a16, a17, a18, and a19 segmented by the first gain correction map segment unit 111b.

In this embodiment, smoothing is performed by averaging the values of the in-plane distribution information in the respective regions a11, a12, a13, a14, a15, a16, a17, a18, and a19, and calculating the averaged values.

Note that the first gain correction map m1 is a map generated under the high tube voltage X-ray condition, and as described in the findings described in the section of “Means for Solving the Problems”, ring-like artifacts occur in the first gain correction map m1. Therefore, the central region a11 may be further segmented into a plurality of regions in accordance with the ring-like artifacts. Further, the shape of the region which are a segmentation target is not limited to a rectangular shape as shown in FIG. 8, and may be, for example, a shape along a ring-like artifact.

Further, the values of the smoothed in-plane distribution information are normalized to acquire final in-plane distribution information. For example, the values of the in-plane distribution information smoothed in each of the regions a11, a12, a13, a14, a15, a16, a17, a18, and a19 shown in FIG. 8 are normalized by dividing by the value of the in-plane distribution information smoothed in the center region a11. In this case, the value of the in-plane distribution information smoothed in the central area a11 is normalized to “1”. Note that Step S1 corresponds to the first gain correction map generation step in the present invention.

### (Step S2) Second Gain Correction Map Formation

In the same manner as in Step S1, the irradiation field moving mechanism 8 moves the slit-like irradiation field in the body axis direction (longitudinal direction) by moving the X-ray tube 2 and the collimator 5 in the body axis direction (longitudinal direction) in a state in which the FPD 3 is fixed as shown in FIG. 6, without placing a subject M on the top board 1.

In Step S2, the X-ray tube 2 emits X-rays under the low tube voltage X-ray condition each time the irradiation field is moved by the irradiation field moving mechanism 8, and the FPD 3 detects the X-rays of the slit-like irradiation field formed by the collimator 5 in a state in which no subject is present. In the same manner as in Step S1, the filter is switched to the metal filter 42 (see FIG. 2) for a low voltage mode in synchronization with the exposure signal under the low tube voltage X-ray condition. This low voltage mode metal filter 42 makes it possible to narrow down the energy spectrum of the X-rays to a certain energy component.

A second gain correction map having in-plane distribution information represented by a two-dimensional distribution of the data output from the FPD 3 at the detection surface in a state in which no subject is present is generated. In the same manner as in Step S1, this second gain correction map is a shape corresponding to the slit-like irradiation field, and what is needed is a single map corresponding to the entire surface of the FPD 3. Therefore, each time the irradiation field is moved by the irradiation field moving mechanism 8, a slit-like second gain correction map corresponding to the slit-like irradiation field is generated, and the second gain correction map coupling unit 112a (see FIG. 4) couples the slit-like second gain correction maps in the body axis direction (longitudinal direction). By coupling in this manner, a single second gain correction map corresponding to the entire surface of the FPD 3 is generated.

In the same manner as in Step S1, the slit-like second gain correction maps are coupled in the body axis direction (longitudinal direction) using a maximum-projection method (MIP) to generate a single second gain correction map corresponding to the entire surface of the FPD 3. In the same manner as in Step S1, the slit-like second gain correction maps may be coupled by weighting addition according to the distance of the center positions of the overlapped maps as in the prior art.

In the same manner as in Step S1, the second gain correction map segment unit 112b (see FIG. 4) segments the second gain correction map m2 into a plurality of regions (e.g., two regions a21 and a22 in FIG. 9). The second gain correction map correction unit 112c (see FIG. 4) corrects the second gain correction map m2 by smoothing the values of the in-plane distribution information in each of the regions segmented by the second gain correction map segment unit 112b. In the same manner as in FIG. 8, in FIG. 9, the values of the in-plane distribution information in each of the regions a21 and a22 are averaged to acquire the averaged value, thereby performing smoothing.

Note that the second gain correction map m2 is a map generated under the low tube voltage X-ray condition, and as described in the findings in the section of “Means for Solving the Problems”, unevenness occur between the upper region a21 and the lower region a22, but ring-like artifacts do not occur as in the case of the map (first gain correction map m1) generated under the high tube voltage X-ray condition. Therefore, the shapes and the numbers of regions which are segment targets are not required to be the same between the first gain correction map and the second gain correction map. Further, the shape of the region which is a segment target is not limited to a rectangular as shown in FIG. 9 in the same manner as in FIG. 8.

Further, in the same manner as in Step S1, the values of the smoothed in-plane distribution information are normalized to acquire final in-plane distribution information. For example, the value of the in-plane distribution information smoothed in each of the regions a21 and a22 shown in FIG. 9 is normalized by dividing by the value of the in-plane distribution information smoothed in the upper region a21. In this case, the value of the in-plane distribution information smoothed in the upper region a21 is normalized to “1”. Note that Step S2 corresponds to the second gain correction map generation step in the present invention.

Note that in FIG. 5, a single first gain correction map corresponding to the entire surface of the FPD 3 was generated by coupling the slit-like first gain correction maps generated under the high tube voltage X-ray condition each time the irradiation field is moved by the irradiation field moving mechanism 8 in the body axis direction (longitudinal direction) (Step S1), and then a single second gain correction map corresponding to the entire surface of the FPD 3 was generated by coupling the slit-like second gain correction maps generated under the low tube voltage X-ray condition each time the irradiation field is moved by the irradiation field moving mechanism 8 in the body axis direction (longitudinal direction) (Step S2); however, the present invention is not limited to the processing of FIG. 5. Step S1 may be performed after Step S2.

Further note that the generation of a single first gain correction map (Step 1) and the generation of a single second correction map (Step 2) may be performed simultaneously. In the generation of a single first gain correction map (Step S1), the slit-like irradiation field is moved in the body axis direction (longitudinal direction) by the irradiation field moving mechanism 8 while alternately applying a high voltage and a low voltage to the X-ray tube 2, the slit-like first gain correction map/the slit-like second gain correction map is alternately generated each time the irradiation field is moved by the irradiation field moving mechanism 8, and the slit-like first gain correction maps are coupled in the body axis direction (longitudinal direction) to generate a single first gain correction map corresponding to the entire surface of FPD 3. In the generation of a single second correction map (Step 2), the slit-like second gain correction maps are coupled in the body axis direction (longitudinal direction) to generate a single second gain correction map corresponding to the entire surface of the FPD 3.

### (Step S3) High Voltage Image Generation/Low Voltage Image Generation

By moving the X-ray tube 2 and the collimator 5 in the body axis direction (longitudinal direction) in a state in which a subject M is placed on the top board 1 and the FPD 3 is fixed as shown in FIG. 6, the irradiation field moving mechanism 8 moves the slit-like irradiation field in the body axis direction (longitudinal direction).

A high voltage of the same value applied at the time of generating the first gain correction map (Step S1) and a low voltage of the same value applied at the time of generating the second gain correction map (Step S2) are alternately applied to the X-ray tube 2. By irradiating the subject M with the X-rays from X-ray tube 2 in a state in which the high voltage and the low voltage are alternately applied to the X-ray tube 2, the generation of the high voltage image by the high voltage image generation unit 113 (see FIG. 4) and the generation of the low voltage image by the low voltage image generation unit 114 (see FIG. 4) are performed simultaneously.

Each time the irradiation field is moved by the irradiation field moving mechanism 8, the X-ray tube 2 emits X-rays under the high tube voltage X-ray condition, the FPD 3 detects the X-rays of the slit-like irradiation field formed by the collimator 5 and transmitted through the subject M, the X-ray tube 2 emits X-rays under the low tube voltage X-ray condition, and the FPD 3 detects the X-rays of the slit-like irradiation field formed by the collimator 5 and transmitted through the subject M. The metal filter 41 for a high voltage mode and the metal filter 42 for a low voltage mode are alternately switched in synchronization with the X-ray irradiation (exposure) from the X-ray tube 2 by the switching between the high tube voltage and the low tube voltage. These metal filters 41 and 42 make it possible to narrow down the energy spectrum of the X-rays to a particular energy component.

The FPD 3 detects the X-rays emitted from the X-ray tube 2 under the high tube voltage X-ray condition and transmitted through the subject M, and a high voltage image captured by the FPD 3 is generated, while the FPD 3 detects the X-rays emitted from the X-ray tube 2 under the low tube voltage X-ray condition and transmitted through the subject M, a low voltage image captured by the FPD 3 is generated. In the same manner as in Step S1 and Step S2, these high voltage image and low voltage image have shapes each corresponding to the slit-like irradiation field, and what is required is a single image corresponding to the entire surface of the FPD 3.

Therefore, each time the irradiation field is moved by the irradiation field moving mechanism 8, a slit-like high voltage image/a slit-like low voltage image corresponding to the slit-like irradiation field is generated, the high voltage image coupling unit 113a (see FIG. 4) couples the slit-like high voltage images in the body axis direction (longitudinal direction), and the low voltage image coupling unit 114a (see FIG. 4) couples the slit-like low voltage images in the body axis direction (longitudinal direction). By coupling in this manner, a single high voltage image/a single low voltage image each corresponding to the entire surface of the FPD 3 is generated.

In the same manner as in Step S1 and Step S2, the slit-like high voltage images/the slit-like low voltage images are coupled in the body axis direction (longitudinal direction) using a maximum-value projection method, thereby generating a single high voltage image/a single low voltage image corresponding to the entire surface of the FPD 3. In the same manner as in Step S1 and Step S2, the slit-like high voltage images/the low voltage images may be coupled by weighted addition according to the distance of the center positions of the overlapped images as in the related art. Note that Step S3 corresponds to the high voltage image generation step and the low voltage image generation step in the present invention.

### (Step S4) First Gain Correction/Second Gain Correction

The first gain correction unit 115 (see FIG. 4) performs the gain correction of the high voltage image generated in Step S3 using the first gain correction map m1 generated in Step S1, and the second gain correction unit 116 (see FIG. 4) performs the gain correction of the low voltage image generated in Step S3 using the second gain correction map m2 generated in Step S2.

Specifically, the pixel value of the high voltage image after the gain correction is acquired by dividing the pixel value of the gain correction target pixel in the high voltage image by the value of the sensitivity of the FPD 3 at the position in the first gain correction map m1 corresponding to the target pixel (in this embodiment, the value of the smoothed and normalized sensitivity). In the same manner, the pixel value of the low voltage image after the gain correction is acquired by dividing the pixel value of the gain correction target pixel in the low voltage image by the value of the sensitivity of the FPD 3 at the position in the second gain correction map m2 corresponding to the target pixel (in this embodiment, the value of the smoothed and normalized sensitivity).

Note that Step S4 corresponds to the first gain correction step and the second gain correction step in the present invention.

### (Step S5) Subtraction Processing

The subtraction processing unit 117 (see FIG. 4) subtracts the high voltage image after the gain correction in Step S4 and the low voltage image after the gain correction in Step S4 to generate an image (subtraction image) after the subtraction processing. The specific calculation method of the subtraction image and the principle of the DXA method will be described with reference to FIG. 10 and p. 20, 22 of Non-Patent Document 1.

X-rays are considered as photons. In a human body, a thickness of a bone cannot be measured because there is a soft tissue around a bone and the incoming photons are attenuated in both the soft tissue and the bone. In cases where the measurement object is a radius or a calcaneus thinner than a trunk, the measurement using a single energy X-ray beam called the SXA (Single Energy X-Ray Absorptiometry) method (also referred to as “SEXA method”) is performed. However, in cases where the measurement object is a lumbar spine or a femur, the measurement is performed by a DXA method.

As shown in FIG. 10, let I0 be the number of incident photons, I be the number of outgoing photons, Tb be a thickness of a bone, and Ts be a thickness of a soft tissue. The unit of the thickness Tb and Ts is [cm]. Further, let the mass attenuation coefficient of a bone be μmb, the mass attenuation coefficient of a soft tissue be μms, a density of a bone be ρb, and a density of a soft tissue be ρs. The unit of the mass attenuation coefficient μmb, μms is [cm²/g], and the unit of the density ρb, ρs, is [g/cm³]. Since a bone is covered with a soft tissue, the attenuation equation of the following equation (1) is established.

I=I₀·e^{-μms·ρs·Ts}·e^{-μmb·ρb·Tb} (1)

In the DXA method, in order to remove the effect of attenuation in a soft tissue, the attenuation equations of the following equations (2) and (3) acquired by transforming the above equation (1) using two kinds of X-rays having different energies are established, and the simultaneous equations are solved.

I₁=I₀₁·e^{-μms}^{₁}^{·ρs·Ts}·e^{-μmb}^{₁}^{·ρb·Tb} (2)

I₂=I₀₂·e^{-μms}^{₂}^{·ρs·Ts}·e^{-μmb}^{₂}^{·ρb·Tb} (3)

When the thickness Ts of a soft tissue is excluded from the simultaneous equations of the above equations (2) and (3), the thickness Tb of the bone is expressed by the following equation (4).

$${T}_{b} = \frac{ln ( {I}_{1} / {I}_{01} ) \cdot μ {ms}_{2} - ln ( {I}_{2} / {I}_{02} ) \cdot μ {ms}_{1}}{ρ b ( μ {mb}_{2} \cdot μ {ms}_{1} - μ {mb}_{1} \cdot μ {ms}_{2} )} \qquad ( 4 )$$

Assuming that the bone quantity of the measurement point is Mb, the bone quantity Mb of the measurement point is expressed by the equation Mb=Tb×ρb. The unit of the bone quantity Mb of the measuring point is [g/cm²]. When the equation (Mb=Tb×ρb) is substituted into the equation (4), bone quantity Mb of the measuring point is expressed by the following equation (5).

$${M}_{b} = \frac{ln ( {I}_{1} / {I}_{01} ) \cdot μ {ms}_{2} - ln ( {I}_{2} / {I}_{02} ) \cdot μ {ms}_{1}}{μ {mb}_{2} \cdot μ {ms}_{1} - μ {mb}_{1} \cdot μ {ms}_{2}} \qquad ( 5 )$$

As is apparent from the above equations (4) and (5), the pixel value of the subtraction image is expressed by the difference value by the difference processing unit 117e (see FIG. 4) of a value acquired by multiplying the value acquired by logarithmically converting the pixel value of the gain correction after the high voltage image by the logarithmic conversion unit 117a (see FIG. 4) by the weighting factor according to the weighting processing unit 117c (see FIG. 4) and the value acquired by multiplying the value acquired by logarithmically converting the pixel value of the low voltage image after the gain correction by the logarithmic conversion unit 117b (see FIG. 4) by the weighting factor by the weighting processing unit 117d (see FIG. 4).

It should be noted that the weighting coefficients used for calculating the pixel values of the subtraction image differ from the coefficients in the equations (4) and (5) since the equation (4) is an equation for calculating the thickness Tb of a bone and the equation (5) is an equation for calculating a bone quantity Mb of a measuring point.

Note that Step S5 corresponds to the subtraction processing step in the present invention.

### (Step S6) Long Image Generation

In cases where the measurement area exceeds the size of the FPD 3, the long image generation unit 118 (see FIG. 4) generates a long image (long subtraction image). Specifically, the imaging system composed of the X-ray tube 2 and the FPD 3 is moved in the body axis direction (longitudinal direction).

In order to relatively move the imaging system composed of the X-ray tube 2 and the FPD 3 with respect to the subject M in the body axis direction (longitudinal direction), in this embodiment, the imaging system moving mechanism 9 (see FIG. 1) moves the imaging system composed of the X-ray tube 2 and the FPD 3 in the body axis direction (longitudinal direction) in a state in which the top board 1 on which a subject M is placed is fixed.

The subtraction images by the subtraction processing unit 117 generated each time the imaging system is moved by the imaging system moving mechanism 9 are coupled in the body axis direction (longitudinal direction). By coupling in this way, a long image (long subtraction image) is generated.

### (Step S7) Bone Density Measurement

The bone density measurement unit 119 (see FIG. 4) performs the bone density measurement by the long image generated in Step S6. The specific calculation method of a bone density (calculation method of a bone mineral quantitative analysis by a DXA method) and the measurement principle of the DXA method will be described with reference to FIGS. 11A, 11B, 12A and 12B and p. 23 to 24 of Non-Patent Document 1.

The measurement principle of the DXA method is schematically represented as a profile curve of the measurement site with low energy and high energy as shown in FIG. 11A (the high energy is represented as “High energy” and the low energy is represented as “Low energy” in FIG. 11A). As shown in FIG. 11A, both of these profile curves are substantially constant in a soft tissue region (denoted as “Soft tissue” in FIG. 11A), and have bimodal shapes downward in a bone quantity of the region through which X-rays are transmitted in the bone region (denoted as “Bone” in FIG. 11A). As shown in FIG. 11A and FIG. 11B, the degree of attenuation of the low energy and the high energy is larger in the low energy, and the difference is further increased in the bone region.

In the DXA method, the baselines (soft tissue regions) of these two profile curves are mathematically matched as shown in FIG. 11B, so that the profile curve at the baseline becomes mathematically “0”. Next, the difference between the two is taken as a profile curve for calculation. The profile curve for the calculation is used to calculate the bone mineral content (bone mineral content) in the DXA method.

First, in the calculation of the bone mineral quantification (bone mineral content) in the DXA method, the flat both ends of the profile curve of the bone quantity Mb in one cross-section are set as a baseline (denoted as “Baseline” in FIGS. 12A and 12B), and the bone edge is determined based on the average value. As a method of determining the bone edge, a differential method, a percentage method, or a threshold method is used. Here, as shown in FIG. 12A, the maximum value of the bone quantity Mb at the measuring point in the profile curve is defined as Mbmax. The baseline average and the Mbmax are used in the percentage method. The specific method of the differential method, the percentage method, and the threshold method are omitted here.

Once the two bone edges are determined, the distance between them is determined as the bone width (denoted as “Bone width” in FIG. 12B). For the determined bone width, the area under the profile curve is calculated by the piecewise quadrature method using the bone quantity Mb of the respective measurement point and the distance ΔX between the measurement points (see FIG. 12B). That is, the strip-like areas formed by Mb and ΔX are integrated over the bone width. Assuming that the value of one cross-sectional acquired by integrating the strip-like area over the bone width is “line BMC”, the value (line BMC) of one cross-section is acquired by the following equation (6).

Line BMC=(½Mb1+Mb2+ . . . +Mbn−1+½Mbn)×ΔX (6)

The unit of the value (line BMC) of one cross-section is [g/cm]. The bone mineral content (BMC: Bone Mineral Content) is acquired by subjecting the value (line BMC) of one cross-section to further a sectional measurement in the body axis direction (Y). The unit of the bone mineral content BMC is [g]. As shown in FIG. 12B, when the measuring range in the body axis direction is defined as LBMC1, LBMCn and the range is segmented by (n−1) at intervals of ΔY, the bone mineral content BMC is acquired by the following equation (7).

BMC=(½LBMC1+LBMC2+ . . . +LBMCn−1+½LBMCn)×ΔY (7)

By dividing the bone mineral content BMC acquired from the above equation (7) by the bone area (Area [cm²]), the bone density (BMD: Bone Mineral Density) which is the area density is acquired. The unit of the bone density BMD is [g/cm²].

According to the bone density measuring apparatus of this embodiment, the flat panel X-ray detector (FPD) 3 detects the X-rays emitted from the X-ray tube 2 under the high tube voltage X-ray condition which is a high voltage condition in which a high voltage (140 kV) is applied to the X-ray tube 2 in a state in which no subject is present, thereby generating a first gain correction map having in-plane distribution information represented by a two-dimensional distribution at the detection surface of the data output from the FPD 3.

In the same manner, the FPD 3 detects the X-rays emitted from the X-ray tube 2 under the low tube voltage X-ray condition which is a low voltage condition in which a low voltage (100 kV) lower than the high voltage is applied to the X-ray tube 2 in a state in which no subject is present, thereby generating a second gain correction map having in-plane distribution information represented by a two-dimensional distribution at the detection surface of the outputted data. The first gain correction map/the first gain correction map is generated as a map having in-plane distribution information acquired by imaging under the high tube voltage X-ray condition/the low tube voltage X-ray condition in a state in which no subject is present.

Then, the FPD 3 detects the X-rays emitted from the X-ray tube 2 under the high tube voltage X-ray condition in which a high voltage of the same value as the high voltage (140 kV) applied to the X-ray tube 2 at the time of generating the first gain correction map and transmitted through the subject M is applied to the X-ray tube 2 to produce the high voltage image captured by the FPD 3.

In the same manner, the FPD 3 detects the X-rays emitted from X-ray tube 2 under the low tube voltage X-ray condition in which a low voltage (100 kV) of the same value as the low voltage applied to X-ray tube 2 at the time of generating the second gain correction map is applied to the X-ray tube 2 and transmitted through the subject M, the low voltage image captured by the FPD 3 is generated. In this manner, at the time of imaging the bone density, a high voltage/a low voltage having the same values as that at the time of generating the first gain correction map/the second gain correction map is applied to the X-ray tube 2 and the subject M is irradiated with the X-rays from the X-ray tube 2, and the high voltage image/the low voltage image is generated.

By performing the gain correction of the high voltage image using the first gain correction map and performing the gain correction of the low voltage image using the second gain correction map, in the high voltage image/the low voltage image after the gain correction under the high tube voltage X-ray condition/the low tube voltage X-ray condition, it is possible to suppress unevenness and artifacts caused by unevenness. Accordingly, it is possible to suppress unevenness and artifacts caused by unevenness also in the image (subtraction image) after the subtraction processing acquired by subtracting the high voltage image after the gain correction/the low voltage image after the gain correction.

FIGS. 14A and 14B are subtraction images acquired by imaging a phantom simulating a bone. FIG. 14A is a subtraction image when the gain correction of the present invention has not been performed. FIG. 14B is a subtraction image when the gain correction of the present invention has been performed. As described in the findings in the section of “Means for Solving the Problems”, the ring-like artifact as shown by the dotted line in FIG. 14A is emphasized in the subtraction image when the gain correction has not been performed. On the other hand, it was confirmed from FIG. 14B that the ring-like artifacts did not appear in the subtraction image when the gain correction has been performed.

As a result, the accuracy of the bone density analysis can be improved by performing the gain correction of the high voltage image/the low voltage image using the first gain correction map/the second gain correction map having in-plane distribution information under the high tube voltage X-ray condition/the low tube voltage X-ray condition and subtracting the high voltage images/the low voltage images after the gain correction.

In this embodiment, the controller 12 has a function of acquiring a reference gain correction map that acquires a reference gain correction map having initially set (default) in-plane distribution information and is provided with a memory unit 13 for storing the reference gain correction map. Further, it is preferable that the input unit 14 has a usage condition switching function for switching the usage conditions of the gain correction map. At the time of the normal X-ray imaging, the reference gain correction map stored by the memory unit 13 is used to perform the gain correction of the X-ray image acquired at the time of the normal X-ray imaging. On the other hand, at the time of the bone density imaging in the subtraction by the subtraction processing unit 117, the first gain correction map having in-plane distribution information under the high tube voltage X-ray condition is used to perform the gain correction of the high voltage image, and the second gain correction map having in-plane distribution information under the low tube voltage X-ray condition is used to perform the gain correction of the low voltage image.

When the normal X-ray imaging other than bone density imaging is performed, in-plane distribution information under the same X-ray condition as that in the normal X-ray imaging is initialized, and the reference gain correction map having the initially set in-plane distribution information is acquired and stored in the memory unit 13. In summary, at the time of the normal X-ray imaging, the reference gain correction map stored in the memory unit 13 is used to perform the gain correction of the X-ray image acquired at the time of the normal X-ray imaging, and at the time of the bone density imaging, the usage condition of the gain correction map is switched so as to perform the gain correction of the high voltage image/the low voltage image using the first gain correction map/the second gain correction map having in-plane distribution information under the high tube voltage X-ray condition/the low tube voltage X-ray condition. As described above, without using a dedicated bone density measuring apparatus, it is possible to perform a common X-ray imaging to which the gain correction is applied and the bone density imaging to which the gain correction is applied, using a normal X-ray imaging apparatus.

In this embodiment, in order to narrow down from the energy spectrum of X-rays to a specific energy component, the high tube voltage X-ray condition (high voltage condition in which a high voltage is applied to the X-ray tube 2) and a low tube voltage X-ray condition (voltage condition in which a low voltage is applied to the X-ray tube 2) and two types of metal filters 41, 42, one for a high voltage mode and the other for a low voltage mode, are combined. That is, these metal filters 41 and 42 are provided so that these metal filters can be switchably arranged on the irradiation side of the X-ray tube 2. The first gain correction map is generated by detecting the X-rays emitted from the X-ray tube 2 in a state in which the metal filter 41 for a high voltage mode is arranged on the irradiation side of the X-ray tube 2 under the high tube voltage X-ray condition in a state in which no subject is present.

In the same manner, the FPD 3 detects the X-rays emitted from the X-ray tube 2 in a state in which the metal filter 42 for a low voltage mode is arranged on irradiation side of the X-ray tube 2 under the voltage X-ray condition in a state in which no subject is present, and the second gain correction map is generated. Then, the FPD 3 detects the X-rays emitted from the X-ray tube 2 under the high tube voltage X-ray condition and transmitted through the subject M in a state in which the metal filter 4 for a high voltage mode is arranged on the irradiation side of the X-ray tube 2, and the high voltage image is generated.

In the same manner, the FPD 3 detects the X-rays emitted from the X-ray tube 2 under the low tube voltage X-ray condition and transmitted through the subject M in a state in which the metal filter 42 for a low voltage mode is arranged on the irradiation side of the X-ray tube 2, and the low voltage image is generated.

In the bone density imaging, it is preferable to apply an operation method called “slot imaging” in which a single X-ray image is generated by coupling a plurality of X-ray images captured by the slit-like X-ray irradiation field in the body axis direction of the subject M. Specifically, the bone density measuring apparatus according to this embodiment is provided with the collimator 5 for forming a slit-like irradiation field by limiting the irradiation region of the X-rays emitted from the X-ray tube 2 and an irradiation field moving mechanism 8 for relatively moving the slit-like irradiation field in the body axis direction with respect to the FPD 3 by relatively moving the X-ray tube 2 and the collimator 5 in the body axis direction of the subject M with respect to the FPD 3.

In this embodiment, the slit-like irradiation field is moved in the body axis direction in a state in which the FPD 3 is fixed by moving the X-ray tube 2 and the collimator 5 in the body axis direction in a state in which the FPD 3 is fixed.

The slit-like first gain correction maps each corresponding to the slit-like irradiation field are coupled in the body axis direction, wherein the slit-like first gain correction maps are formed by detecting the X-rays of the slit-like irradiation unit irradiated from the X-ray tube under the high tube voltage X-ray condition and formed by the collimator 5 in a state in which no subject is present each time the irradiation field is moved by the irradiation field moving mechanism 8. By coupling in this manner, a single first gain correction map corresponding to the entire surface of the FPD 3 is generated.

In the same manner, the slit-like second gain correction maps each corresponding to the slit-like irradiation field are coupled in the body axis direction, wherein the slit-like second gain correction maps are formed by detecting the X-rays of the slit-like irradiation unit irradiated from the X-ray tube under the low tube voltage X-ray condition and formed by the collimator 5 in a state in which no subject is present each time the irradiation field is moved by the irradiation field moving mechanism 8 by the FDP 3. By coupling in this manner, a single second gain correction map corresponding to the entire surface of the FPD 3 is generated.

The slit-like high voltage images each corresponding to the slit-like irradiation field, which are generated by detecting the X-rays of the slit-like irradiation field emitted from the X-ray tube 2 under the high tube voltage X-ray condition and formed by the collimator 5 and transmitted through subject M each time the irradiation field is moved by the irradiation field moving mechanism 8, are coupled in the body axis direction. By coupling in this manner, a single high voltage image corresponding to the entire surface of the FPD 3 is generated.

In the same manner, the slit-like low voltage images each corresponding to the slit-like irradiation field, which are generated by detecting the X-rays of the slit-like irradiation field emitted from the X-ray tube 2 under the low tube voltage X-ray condition and formed by the collimator 5 and transmitted through subject M each time the irradiation field is moved by the irradiation field moving mechanism 8, are coupled in the body axis direction. By coupling in this manner, a single low voltage image corresponding to the entire surface of the FPD 3 is generated.

When emitting the X-rays on the entire surface of the FPD 3 without limiting the irradiation region of X-rays, since the X-rays are incident on the end portion of the FPD 3 from the oblique direction, distortions occur in the X-ray image at the end portion. On the other hand, since the X-rays are incident perpendicularly on the detection surface of the FPD 3 by the slot imaging, distortions of the X-ray image (first gain correction map/second gain correction map, high voltage image/low voltage image) can be suppressed. Further, due to the slit-shape, it is possible to acquire an X-ray image (first gain correction map/second gain correction map, high voltage image/low voltage image) of high image quality in which the effect of scattered radiation is suppressed.

Further provided are an imaging system moving mechanism 9 for relatively moving the imaging system composed of the X-ray tube 2 and the FPD 3 in the body axis direction with respect to the subject M and a long image generation unit 118 for generating a long image by coupling the images (subtraction image) after the subtraction processing, which are generated each time the imaging system is moved by the imaging system moving mechanism 9.

In this embodiment, the imaging system composed of the X-ray tube 2 and the FPD 3 is moved in the body axis direction in a state in which the top board 1 on which a subject M is placed is fixed. In this embodiment, the bone density imaging is preferably applied to the “long imaging” for generating a long image in which a plurality of X-ray images is coupled in the body axis direction (wider than the detecting region on the entire surface of the FPD 3) in the same manner as in the slot imaging.

As described above, in this embodiment, the slot imaging and the long imaging are combined. Specifically, slit-like X-ray images are respectively generated while relatively moving the X-ray tube 2 and the collimator 5 in the body axis direction with respect to the FPD 3 in the slot imaging (in this embodiment, while moving the X-ray tube 2 and the collimator 5 in the body axis direction in a state in which the FPD 3 is fixed), and a single X-ray image corresponding to the entire surface of the FPD 3 is generated by coupling these X-ray images in the body axis direction, and then a single X-ray image corresponding to the entire surface is repeatedly generated by the slot imaging while relatively moving the imaging system in the body axis direction with respect to the subject M (in this embodiment, while moving the top board 1 on which a subject M is placed in the body axis direction in a state in which the imaging system is fixed). Then, a single X-ray image corresponding to the entire surface of the FPD 3 is generated by slot imaging. This is repeated. Then, a long image is generated by coupling the generated X-ray images in the body axis direction.

In this embodiment, a long subtraction image is generated as a long image by coupling the images (subtraction images) each generated after the subtraction processing in the body axis direction.

As described in the section of “Means for Solving the Problems”, it is considered that the accuracy can be improved if the gain correction is performed for each pixel, but this is not the case in practice. Due to the fluctuations of the pixel value (i.e., statistical errors), the actual pixel value is not a true value. Therefore, if the gain correction of the high voltage image/the low voltage image is performed for each pixel by using a gain correction map which is not a true value, the accuracy may be deteriorated.

For this reason, in this embodiment, provided are a first gain correction map segment unit 111b for segmenting the first gain correction map to a plurality of regions, a second gain correction map segment unit 112b for segmenting the second gain correction map into a plurality of regions, a first gain correction map correction unit 111c for correcting the first gain correction map by smoothing the values of the in-plane distribution information in each region segmented by the first gain correction map segment unit 111b, and a second gain correction map correction unit 112c for correcting the second gain correction map by smoothing the values of the in-plane distribution information in each region segmented by the second gain correction map segment unit 112b.

In this manner, by performing the gain correction of the high voltage image/the low voltage image using the first gain correction map/the second gain correction map corrected by smoothing the values of the in-plane distribution information in each region, the gain correction can be performed appropriately and accurately.

As repeatedly described above, the shapes and numbers of regions which are segment targets are not required to be the same among the first gain correction maps/the second gain correction maps. Further, in this embodiment, an average value is acquired as an example of the smoothing, but the smoothing is not limited to an average value. For example, the smoothing may be performed using the median value or the smoothing may be performed using the mode value. In other words, the smoothing may be performed using statistics.

Further, according to the bone density imaging method of this embodiment, by performing Step S1 (first gain correction map generation), Step S2 (second gain correction map generation), Step S3 (high voltage image generation/low voltage image generation), Step S4 (first gain correction/second gain correction), and Step S5 (subtraction processing) corresponding to the respective steps shown in FIG. 5, the bone density imaging can be suitably performed, which in turn can improve the accuracy of the bone density analysis.

In Step S1 corresponding to the first gain correction map generation step, the slit-like irradiation fields each corresponding to the slit-like irradiation field, which are generated by detecting the X-rays of the slit-like irradiation field emitted from the X-ray tube 2 under the high tube voltage X-ray condition and formed by the collimator 5 each time the slit-like irradiation field is relatively moved in the body axis direction with respect to the FPD 3 (in this embodiment, each time the slit-like irradiation is moved in the body axis direction in a state in which the FPD is fixed), are coupled in the body axis direction, and a single first gain correction map corresponding to the entire surface of the FPD 3 is generated by coupling the slit-like first gain correction maps.

In the same manner, in the second gain correction map generation step, the slit-like second gain correction maps each corresponding to the slit-like irradiation field, which are generated by detecting the X-rays of a slit-like of the slit-like irradiation field emitted from the X-ray tube 2 under the low tube voltage X-ray condition and formed by the collimator 5 in a state in which no subject is present by the FPD 3 each time the slit-like irradiation field is moved in the body axis direction with respect to the FPD 3 (in this embodiment, each time the slit-like irradiation field is moved in the body axis direction in a state in which the FPD is fixed), are coupled in the body axis direction, and the slit-like second gain correction maps is coupled in the body axis direction to generate a single second gain correction map corresponding to the entire surface of the FPD 3.

In the case of applying the slot imaging as in this embodiment, the above-described modes are conceivable.

That is, Step S2 corresponding to the second gain correction map generation step is performed after Step S1 corresponding to the first gain correction map generation step. Alternatively, Step S1 corresponding to the first gain correction map generation step is performed after Step S2 corresponding to the second gain correction map generation step. In this manner, Step S1 corresponding to the first gain correction map generation step and Step S2 corresponding to the second gain correction map generation step may be performed in a temporally separated manner.

On the other hand, the slit-like irradiation field is relatively moved in the body axis direction with respect to the FPD 3 (in this embodiment, the slit-like irradiation field is moved in the body axis direction in a state in which the FPD 3 is fixed) while alternately applying the high voltage and the low voltage to the X-ray tube 2, and the slit-like first gain correction map and the second gain correction map are alternately generated each time the slit-like irradiation field is relatively moved in the body axis direction with respect to the FPD 3 (in this embodiment, each time the slit-like irradiation field is moved in the body axis direction in a state in which the FPD 3 is fixed). Then, the first gain correction map generation step of generating a single first gain correction maps each corresponding to the entire surface of the FPD 3 by coupling the slit-like first gain correction maps in the body axis direction (Step S1) and the second gain correction map generation step for generating a single second gain correction map corresponding to the entire surface of the FPD 3 by coupling the slit-like second gain correction maps in the body axis direction may be performed simultaneously.

As described above, by relatively moving the slit-like irradiation field in the body axis direction with respect to FPD3 while alternately applying the high voltage and the low voltage to the X-ray tube 2 (by moving the slit-like irradiation field in the body axis direction in a state in which the FPD 3 is fixed), the first gain correction map generation step (Step S1) and the second gain correction map generation step (Step S2) may be performed.

In this embodiment, it is preferable to simultaneously perform Step S3 corresponding to the high voltage image generation step for generating a high voltage image and the low voltage image generation step for generating a low voltage image by irradiating the subject M with the X-rays from the X-ray tube 2 while alternately applying a high voltage having the same value as that at the time of generating the first gain correction map and a low voltage having the same value as that at the time of generating the second gain correction map to the X-ray tube 2. With this, the high voltage image and the low voltage image can be simultaneously acquired in one imaging including the slot imaging and the long imaging as in this embodiment.

The present invention is not limited to the above-described embodiment, and can be modified as described below.

(1) In the above-described embodiment, in order to narrow down from the energy spectrum of X-rays to a specific energy component, the high voltage condition (the high voltage condition in which a high voltage is applied to the X-ray tube 2) and the low voltage condition (the low voltage condition in which a low voltage is applied to the X-ray tube 2) and two types of metal filters 41 and 42, one for a high voltage mode and the other for a low voltage mode, are combined, but it is not always required to provide the metal filters 41 and 42.

(2) In the above-described embodiment, the high voltage image and the low voltage images are simultaneously acquired by single imaging including the slot imaging and the long imaging by irradiating the subject M with the X-rays from the X-ray tube 2 by alternately applying a high voltage and a low voltage to the X-ray tube 2, but the present invention is not limited to such an operation method. In the slot imaging or the long imaging, the low voltage image may be generated after the generation of the high voltage image, or the high voltage image may be generated after the generation of the low voltage image.

(3) In the above-described embodiment, the long image is generated by coupling images (subtraction images) after the subtraction processing in the body axis direction in the long imaging, but the subtraction processing may be performed after coupling the images in the body axis direction. That is, the long image may be generated by performing the subtraction of long images of the long high voltage image/the long low voltage image after generating the long high voltage image/the low voltage image by coupling the high voltage images/the low voltage images in the body axis direction.

(4) In the above-described embodiment, the slot imaging is performed, but when the X-rays are incident on the end portion of the detector substantially perpendicularly when a small-sized detector is used, the single first gain correction map/the second gain correction map or the high voltage image/the low voltage image corresponding to the entire surface of the detector may be directly generated without coupling the first gain correction map/the second gain correction map or the high voltage image/the low voltage image in the body axis direction.

(5) In the above-mentioned embodiment, the long image (long subtraction image) is generated when the measurement area exceeds the size of the FPD 3, but the long imaging is not necessarily performed when the measurement area is equal to or smaller than the size of the FPD 3.

(6) In the above-described embodiment, the slit-like irradiation field is relatively moved in the body axis direction with respect to the detector (FPD 3) by relatively moving the X-ray tube 2 and the collimator 5 in the body axis direction with respect to the detector (FPD 3) in a state in which the detector (FPD 3) is fixed in the slot imaging, but the mode of moving is not limited to the above. For example, the slit-like irradiation field may be relatively moved in the body axis direction with respect to the detector (FPD 3) in a state in which the X-ray tube 2 and the collimator 5 are fixed by moving the detector (FPD 3) in the body axis direction in a state in which the X-ray tube 2 and the collimator 5 are fixed. Alternatively, the slit-like irradiation field may be relatively moved in the body axis direction with respect to detector (FPD3) by moving the detector (FPD 3) and the X-ray tube 2 and the collimator 5 in opposite directions in the body axis direction.

(7) In the above-described embodiment, the imaging system is relatively moved in the body axis direction with respect to the subject M by moving the imaging system in the body axis direction in a state in which the top board 1 on which a subject M is placed is fixed in the long imaging, but the mode of moving is not limited to the above. For example, the imaging system may be relatively moved with respect to the subject M in the body axis direction by moving the top board 1 in the body axis direction in a state in which the imaging system is fixed. Further, the imaging system may also be relatively moved with respect to the subject M in the body axis direction by moving the top board 1 and the imaging system in opposite directions in the body axis direction.

(8) In the above-described embodiment, the usage conditions of the gain correction map are switched at the time of the normal X-ray imaging and at the time of the bone density imaging in the subtraction in order to perform the normal X-ray imaging to which the gain correction is applied and the bone density imaging to which the gain correction is applied by using a common X-ray imaging apparatus without using a dedicated bone density measuring apparatus, but the usage condition of the gain correction map is not always required to be switched when the gain correction is performed by using a dedicated bone density measuring apparatus.

### DESCRIPTION OF SYMBOLS

2: X-ray tube
3: Flat panel X-ray detector (FPD)
4: Filter
41: High voltage mode metal filter
42: Low voltage mode metal filter
5: Collimator
8: Irradiation field moving mechanism
9: Imaging system moving mechanism
11: Image processing unit
111: First gain correction map generation unit
111a: First gain correction map coupling unit
111b: First gain correction map segment unit
111c: First gain correction map correction unit
112: Second gain correction map generation unit
112a: Second gain correction map coupling unit
112b: Second gain correction map segment unit
112c: Second gain correction map correction unit
113: High voltage image generation unit
113a: High voltage image coupling unit
114: Low voltage image generation unit
114a: Low voltage image coupling unit
115: First gain correction unit
116: Second gain correction unit
117: Subtraction processing unit
118: Long image generation unit
12: Controller
13: Memory unit
14: Input unit
R: Irradiation region
M: Subject

1. A bone density measuring apparatus for measuring a bone density by X-ray imaging, comprising:
an X-ray tube configured to emit X-rays;
a detector configured to detect the X-rays emitted from the X-ray tube;
a first gain correction map generation means configured to generate a first gain correction map having in-plane distribution information represented by a two-dimensional distribution at a detection surface of data output from the detector by detecting the X-rays emitted from the X-ray tube by the detector in a state in which no subject is present under a high tube voltage X-ray condition which is a high voltage condition in which a high voltage is applied to the X-ray tube;
a second gain correction map generation means configured to generate a second gain correction map having in-plane distribution information represented by a two-dimensional distribution at a detection surface of data output from the detector by detecting the X-rays emitted from the X-ray tube by the detector in a state in which no subject is present under a low tube voltage X-ray condition which is a low voltage condition in which a low voltage lower than the high voltage is applied to the X-ray tube;
a high voltage image generation means configured to generate a high voltage image captured by the detector by detecting the X-rays emitted from the X-ray tube and transmitted through the subject by the detector under the high tube voltage X-ray condition in which a high voltage of the same value as the high voltage applied to the X-ray tube at the time of generating the first gain correction map by the first gain correction map generation means is applied to the detector;
a low voltage image generation means configured to generate a low voltage image captured by the detector by detecting the X-rays emitted from the X-ray tube and transmitted through the subject by the detector under the low tube voltage X-ray condition in which a low voltage of the same value as the low voltage applied to the X-ray tube at the time of generating the second gain correction map by the second gain correction map generation means is applied to the detector;
a first gain correction means configured to perform a gain correction of the high voltage image generated by the high voltage image generation means using the first gain correction map generated by the first gain correction map generation means;
a second gain correction means configured to perform a gain correction of the low voltage image generated by the low voltage image generation means using the second gain correction map generated by the second gain correction map generation means; and
a subtraction processing means configured to perform a subtraction of the high voltage image after the gain correction by the first gain correction means and the low voltage image after the gain correction by the second gain correction means,
wherein the bone density is measured by an image after subtraction processing by the subtraction processing means.

2. The bone density measuring apparatus as recited in claim 1, further comprising:
a reference gain correction map acquisition means configured to acquire a reference gain correction map having initially set in-plane distribution information;
a reference gain correction map storage means configured to store the reference gain correction map; and
a usage condition switching means configured to switch a usage condition of a gain correction map so that (a) a gain correction of an X-ray image acquired at a time of acquiring normal X-ray imaging using the reference gain correction map stored by the reference gain correction map storage means at a time of normal X-ray imaging and (b) a gain correction of the high voltage image is performed using the first gain correction map having in-plane distribution information under the high tube voltage X-ray condition at a time of bone density imaging by subtraction by the subtraction processing means and a gain correction of the low voltage image is performed using the second gain correction map having in-plane distribution information under the low tube voltage X-ray condition.

3. The bone density measuring apparatus as recited in claim 1, further comprising:
two types of filters including a filter for a high voltage mode and a filter for a low voltage mode, the two types of filters being configured to be alternately arranged on an irradiation side of the X-ray tube;
wherein the first gain correction map generation means generates the first gain correction map by detecting the X-rays emitted from the X-ray tube by the detector in a state in which the filter for a high voltage mode is arranged on the irradiation side of the X-ray tube under the high tube voltage X-ray condition in a state in which no subject is present,
wherein the second gain correction map generation means generates the second gain correction map by detecting the X-rays emitted from the X-ray tube by the detector in a state in which the filter for a low voltage mode is arranged on the irradiation side of the X-ray tube under the low tube voltage X-ray condition in a state in which no subject is present,
wherein the high voltage image generation means generates the high voltage image by detecting the X-rays emitted from the X-ray tube and transmitted through the subject by the detector in a state the filter for a high voltage mode is arranged on the irradiation side of the X-ray tube under the high tube voltage X-ray condition, and
wherein the low voltage image generation means generates the low voltage image by detecting the X-rays emitted from the X-ray tube and transmitted through the subject by the detector in a state in which the filter for a low voltage mode is arranged on the irradiation side of the X-ray tube under the low tube voltage X-ray condition.

4. The bone density measuring apparatus as recited in claim 1, further comprising:
a collimator configured to form a slit-like irradiation field by limiting an irradiation region of the X-rays emitted from the X-ray tube; and
an irradiation field moving mechanism configured to relatively move the slit-like irradiation field in the body axis direction of the subject with respect to the detector by relatively moving the X-ray tube and the collimator in the body axis direction with respect to the detector,
wherein the first gain correction map generation means includes:
a first gain correction map coupling means configured to couple the slit-like first gain correction maps each corresponding to the slit-like irradiation field in the body axis direction, wherein the slit-like gain correction maps are generated by detecting the X-rays of the slit-like irradiation field emitted from the X-ray tube under the high tube voltage X-ray condition and formed by the collimator by the detector in a state in which no subject is present each time the irradiation field is moved by the irradiation field moving mechanism,
a single piece of the first gain correction map corresponding to an entire surface of the detector is generated by coupling the slit-like first gain correction maps with the first gain correction map coupling means,
wherein the second gain correction map generation means includes:
a second gain correction map coupling means configured to couple the slit-like second gain correction maps each corresponding to the slit-like irradiation field in the body axis direction, wherein the slit-like gain correction maps are generated by detecting the X-rays of the slit-like irradiation field emitted from the X-ray tube under the low tube voltage X-ray condition and formed by the collimator by the detector in a state in which no subject is present,
a single piece of the second gain correction map corresponding to an entire surface of the detector is generated by coupling the slit-like second gain correction maps with the second gain correction map coupling means,
wherein the high voltage image generation means includes:
a high voltage image coupling means configured to couple the silt-like high voltage images each corresponding to the silt-like irradiation field in the body axis direction, wherein the slit-like high voltage images are formed by detecting the X-rays of the slit-like irradiation field emitted from the X-ray tube, formed by the collimator and transmitted through the subject under the high tube voltage X-ray condition by the detector each time the irradiation field is moved by the irradiation field moving mechanism,
a single piece of the high voltage image corresponding to an entire surface of the detector by coupling the slit-like high voltage images with the high voltage image coupling means, and
wherein the low voltage image generation means includes:
a low voltage image coupling means configured to couple slit-like low voltage images each corresponding to the slit-like irradiation field in the body axis direction, wherein the slit-like low voltage images are formed by detecting the X-rays of the slit-like irradiation field emitted from the X-ray tube, formed by the collimator, and transmitted through the subject under the low tube voltage X-ray condition by the detector each time the irradiation field is moved by the irradiation field moving mechanism,
a single piece of the low voltage image corresponding to an entire surface of the detector is generated by coupling the slit-like voltage images with the low voltage image coupling means.

5. The bone density measuring apparatus as recited in claim 1, further comprising:
an imaging system moving mechanism configured to relatively move an imaging system composed of the X-ray tube and the detector in the body axis direction with respect to the subject; and
a long image generation means configured to generate a long image by coupling images after subtraction processing by the subtraction processing means in the body axis direction, each of the images being generated each time the imaging system is moved by the imaging system moving mechanism.

6. The bone density measuring apparatus as recited in claim 1, further comprising:
a first gain correction map segment means configured to segment the first gain correction map into a plurality of regions;
a second gain correction map segment means configured to segment the second gain correction map into a plurality of regions;
a first gain correction map correction means configured to correct the first gain correction map by smoothing values of the in-plane distribution information in each region segmented by the first gain correction map segment means; and
a second gain correction map correction means configured to correct the second gain correction map by smoothing values of the in-plane distribution information in each region segmented by the second gain correction map segment means.

7. A bone density measuring method for measuring a bone density using a bone density measuring apparatus equipped with an X-ray tube configured to emit X-rays and a detector configured to detect the X-rays emitted from the X-ray tube; the bone density measuring method comprising:
a first gain correction map generation step for generating a first gain correction map having in-plane distribution information represented by a two-dimensional distribution at a detection surface of data output from the detector by detecting the X-rays emitted from the X-ray tube by the detector in a state in which no subject is present under a high tube voltage X-ray condition which is a high voltage condition in which a high voltage is applied to the X-ray tube;
a second gain correction map generation step for generating a second gain correction map having in-plane distribution information represented by a two-dimensional distribution at a detection surface of data output from the detector by detecting the X-rays emitted from the X-ray tube by the detector in a state in which no subject is present under a low tube voltage X-ray condition which is a low voltage condition in which a low voltage lower than the high voltage is applied to the X-ray tube;
a high voltage image generation step for generating a high voltage image captured by the detector by detecting the X-rays emitted from the X-ray tube and transmitted through the subject by the detector under the high tube voltage X-ray condition in which a high voltage of the same value as the high voltage applied to the X-ray tube at the time of generating the first gain correction map in the first gain correction map generation step is applied to the X-ray tube;
a low voltage image generation step for generating a low voltage image captured by the detector by detecting the X-rays emitted from the X-ray tube and transmitted through the subject by the detector under the low tube voltage X-ray condition in which a low voltage of the same value as the low voltage applied to the X-ray tube at the time of generating the second gain correction map in the second gain correction map generation step is applied to the x-ray tube;
a first gain correction step for performing a gain correction of the high voltage image generated in the high voltage image generation step using the first gain correction map generated in the first gain correction map generation step;
a second gain correction step for performing a gain correction of the low voltage image generated in the low voltage image generation step using the second gain correction map generated in the second gain correction map generation step; and
a subtraction processing step for performing a subtraction of the high voltage image after the gain correction in the first gain correction step and the low voltage image after the gain correction in the second gain correction step,
wherein the bone density is measured by an image after subtraction processing in the subtraction processing step.

8. The bone density imaging method as recited in claim 7,
wherein when performing slot imaging in which a single X-ray image is generated by coupling a plurality of X-ray images each captured by a slit-like X-ray irradiation field in a body axis direction of a subject, the slit-like irradiation field is formed by limiting the irradiation region of the X-rays emitted from the X-ray tube by a collimator, and the slit-like irradiation field is relatively moved in the body axis direction with respect to the detector by relatively moving the X-ray tube and the collimator in the body axis direction with respect to the detector to perform the slot imaging,
wherein in the first gain correction map generation step, the slit-like first gain correction maps each corresponding to the slit-like irradiation field are coupled in the body axis direction, wherein the slit-like first gain correction maps are formed by detecting the X-rays of the slit-like irradiation field emitted from the X-ray tube and formed by the collimator under the high tube voltage X-ray condition in a state in which no subject is present by the detector each time the slit-like irradiation field is relatively moved in the body axis direction with respect to the detector, and a single first gain correction map corresponding to an entire surface of the detector is generated by coupling the slit-like first gain correction maps in the body axis direction,
wherein in the second gain correction map generation step, the slit-like second gain correction maps each corresponding to the slit-like irradiation field are coupled in the body axis direction, wherein the slit-second gain correction maps are formed by detecting the X-rays of the slit-like irradiation field emitted from the X-ray tube and formed by the collimator under the low tube voltage X-ray condition in a state in which no subject is present by the detector each time the slit-like irradiation field is relatively moved in the body axis direction with respect to the detector, and a single second gain correction map corresponding to an entire surface of the detector is generated by coupling the slit-like second gain correction maps in the body axis direction, and
wherein the second gain correction map generation step is performed after the first gain correction map generation step or the first gain correction map generation step is performed after the second gain correction map generation step.

9. The bone density imaging method as recited in claim 7,
wherein when performing slot imaging in which a single X-ray image is generated by coupling a plurality of X-ray images each captured by a slit-like X-ray irradiation field in a body axis direction of a subject, the slit-like irradiation field is formed by limiting the irradiation region of the X-rays emitted from the X-ray tube by a collimator, and the slit-like irradiation field is relatively moved in the body axis direction with respect to the detector by relatively moving the X-ray tube and the collimator in the body axis direction with respect to the detector to perform the slot imaging,
wherein in the first gain correction map generation step, the slit-like first gain correction maps each corresponding to the slit-like irradiation field are coupled in the body axis direction, wherein the slit-like first gain correction maps are formed by detecting the X-rays of the slit-like irradiation field emitted from the X-ray tube and formed by the collimator under the high tube voltage X-ray condition in a state in which no subject is present by the detector each time the slit-like irradiation field is relatively moved in the body axis direction with respect to the detector, and a single first gain correction map corresponding to an entire surface of the detector by coupling the slit-like first gain correction maps in the body axis direction,
wherein in the second gain correction map generation step, the slit-like second gain correction maps each corresponding to the slit-like irradiation field are coupled in the body axis direction, wherein the slit-second gain correction maps are formed by detecting the X-rays of the slit-like irradiation field emitted from the X-ray tube and formed by the collimator under the low tube voltage X-ray condition in a state in which no subject is present by the detector each time the slit-like irradiation field is relatively moved in the body axis direction with respect to the detector, and a single second gain correction map corresponding to an entire surface of the detector by coupling the slit-like second gain correction maps in the body axis direction,
wherein the slit-like irradiation field is relatively moved in the body axis direction with respect to the detector while alternately applying a high voltage and a low voltage to the X-ray tube, and the slit-like first gain correction map and the slit-like second gain correction map are alternately generated each time the slit-like irradiation field is relatively moved in the body axis direction with respect to the detector, and
wherein the first gain correction map generation step for generating a single first gain correction map corresponding to an entire surface of the detector by coupling the slit-like first gain correction maps in the body axis direction and the second gain correction map generation step for generating a single gain correction map corresponding to the entire surface of the detector by coupling the slit-like second gain correction maps in the body axis direction are performed simultaneously.

10. The bone density imaging method as recited in claim 7,
wherein the high voltage image generation step for generating the high voltage image and the low voltage image generation step for generating the low voltage image are performed simultaneously by alternately applying a high voltage having the same value as that at the time of generating the first gain correction map and a low voltage having the same value as that at the time of generating the second gain correction map to the X-ray tube and irradiating the subject with the X-ray tube.
